(19) United States
(12) Patent Application Publication
(10) Pub. No.: US 2024/0352011 A1
(43) Pub. Date: Oct. 24, 2024

(54) Macrocyclic Ligands with Picolinate Group(s), Complexes and Medical Uses Thereof

(71) Applicant: GUERBET, Villepinte (FR)

(72) Inventors: Olivier Rousseaux, Senlis (FR); William Pauly-Batard, Messy (FR); Olivier Fougere, Mortefontaine (FR); Sarah Catoen, Livry Gargan (FR)

(21) Appl. No.: 18/293,079

(22) PCT Filed: Jul. 29, 2022
(86) PCT No.: PCT/EP2022/071321

§ 371 Date: Jan. 29, 2024

(30) Foreign Application Priority Data
Jul. 30, 2021 (EP) 21306070.0

(51) Int. Cl. C07D 471/04; A61K 31/4375; A61K 47/44; A61P 35/00; C07F 1/00; C07F 5/00; C07F 13/00

(52) U.S. Cl. CPC C07D 471/04; A61K 31/4375; A61K 47/44; A61P 35/00; C07F 1/005; C07F 5/003; C07F 13/005

(57) ABSTRACT

The present invention relates to new macrocyclic ligands substituted with at least one picolinate group, to the radioactive complexes thereof and to the uses thereof in medical imaging and/or in therapy, in particular in interventional radiology. The present invention also relates to a new process for preparing ligands according to the invention, and also to the preparation intermediates thereof.

The present invention relates to novel macrocyclic ligands and also to their complexes, in particular radioactive complexes, and to their uses in medical imaging and/or in therapy, in particular in interventional radiology.

The present invention also relates to a novel process for the preparation of ligands such as according to the invention, and also to their preparation intermediates.

The need for targeted and personalized treatments in oncology has led to the development of novel therapeutic strategies based on early detection tools combined with more specific and more efficient vectorized treatments.

Interventional radiology is a very promising direction in individualized medicine. It makes it possible to combine, in one and the same sequence, precise diagnosis of the lesion or tumor and/or its instantaneous treatment, guided and controlled by images. It is described as minimally invasive surgery and can as a result be performed on an outpatient basis, which makes possible a saving of many expensive days of hospitalization for an effectiveness which is often comparable to that of conventional surgery. Interventional radiology can thus represent an alternative or an extension to conventional surgical treatment.

Interventional radiology makes it possible to access a lesion or tumor located inside the body in order to perform a diagnostic action (for example sampling) or a therapeutic action. Imaging by fluoroscopy, echography, scanner or MRI allows pinpointing, guiding and control which is optimum of the medical act.

There thus exists a need for novel molecules which can be used in medical imaging and/or in therapy, in particular in interventional radiology. More particularly, there exists a need for ligands which make it possible to complex chemical elements, in particular metals, so as to obtain complexes which can be used in medical imaging and/or in therapy, in particular in interventional radiology.

Such ligands must in particular be stable in human serum and must complex metals sufficiently strongly for the latter to reach their target and not to diffuse into other sensitive organs or tissues, such as bones, lungs and kidneys.

It is an aim of the present invention to provide novel ligands which make it possible to complex chemical elements, in particular radioelements.

It is another aim of the present invention to provide novel complexes, in particular radioactive complexes.

It is an aim of the present invention to provide ligands and/or complexes which are particularly useful in medical imaging and/or in therapy, in particular in the treatment of cancers.

Another aim of the present invention is to provide a pharmaceutical composition comprising complexes making possible medical imaging, targeting and/or treatment of cancers.

It is an aim of the present invention to provide a novel process for the preparation of these ligands.

Starting from the studies described in WO 2017/109217 and in Le Fur et al. (Inorganic Chemistry, Volume 57, Issue 4, pages 2051-2063, 2018), the inventors have developed novel ligands of high affinity for certain metals, in particular rare earth metals, complexes which are very stable and which exhibit high kinetic inertias. Moreover, these complexes can be easily radiolabeled and then exhibit a satisfactory radiochemical yield and a satisfactory radiochemical purity. These complexes can also be incorporated in an iodinated oil in a stable manner and with good reproducibility, and thus exhibit a biodistribution profile which makes possible their use in the treatment of cancer.

The present invention relates to a compound of following general formula (I):

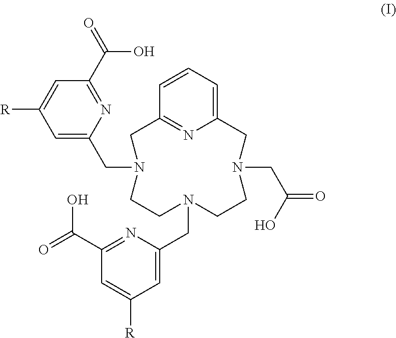

in which R is a group of following formula (II):

—C≡C-Ph-L1-(CH₂)ₙ-L2 (II)
with
   L1 representing Ph or —C≡C— or CH₂,
   L2 representing H or Ph or alkylphenyl,
   n of between 4 and 12,
or one of its pharmaceutically acceptable salts.

According to a preferred embodiment, the present invention relates to a compound of general formula (I) in which the group R is chosen from:
the group R in which L1=CH₂, n=7 and L2=H, which corresponds to the following structure:

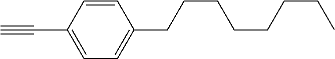 [SMILES: C#Cc1ccc(CCCCCCCC)cc1]

the group R in which L1=Ph, n=8 and L2=H, which corresponds to the following structure:

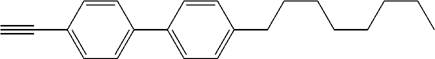 [SMILES: C#Cc1ccc(-c2ccc(CCCCCCCC)cc2)cc1]

the group R in which L1=—C≡C—, n=8 and L2=Ph, which corresponds to the following structure:

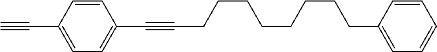 [SMILES: C#Cc1ccc(C#CCCCCCCCCc2ccccc2)cc1]

and one of its pharmaceutically acceptable salts.

The inventors have developed novel ligand-metal complexes (complexes also known as chelates) starting from the pyclen macrocycle (3,6,9,15-tetraazabicyclo[9.3.1]pentadeca-1(15),11,13-triene), variously substituted by acetate and/or picolinate (6-methylene-2-pyridinecarboxylic acid) groups. The pyclen macrocycle has the following formula:

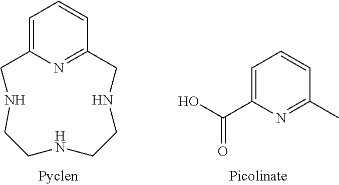

Like the complexes described in WO 2017/109217, the complexes according to the invention exhibit good thermodynamic stability and also good kinetic inertia. They can also be dissolved in an iodinated oil, such as Lipiodol®, an iodinated oil manufactured and sold by Guerbet and which consists of ethyl esters of iodinated fatty acids of poppy
oil. Thus, the complexes according to the invention dissolved in an iodinated oil, such as Lipiodol®, can be vectorized in particular toward the liver and can make it possible to visualize and/or treat cancers, for example cancers of the liver.

These complexes also exhibit a good extraction yield in an iodinated oil, such as Lipiodol®. They exhibit in particular good incorporation of the radioactivity (radiochemical yield) in an iodinated oil, such as Lipiodol®, and good stability of the radioactive solution of Lipiodol® during in vitro tests.

In particular, the combination of the properties of vectorization of Lipiodol®, of therapeutic effectiveness of the radioelements, and the good tolerance of these products make it possible to provide a therapeutic treatment of cancers which is safe and easier to carry out.

The vectorization of the complexes according to the invention by an iodinated oil, such as Lipiodol®, makes it possible in particular to avoid poor delivery of the complexes and thus reduces the risk of adverse effects in healthy organs, especially healthy liver or in extrahepatic organs, and makes it possible to achieve the effective dose of radioactivity in the tumor.

More particularly, this vectorization facilitates the work of the interventional radiologist at the time of the injection of the complexes according to the invention. For example, during an intra-arterial injection monitored by fluoroscopy, the radiologist's act will be more precise and safer, making possible adjustment of the rate of delivery of the complexes as a function of the uptake by the tumor of the complexes according to the invention.

### Definitions

The term “ligand” is understood to mean a compound capable of complexing a chemical element, such as a metal, preferably a radioelement. According to one embodiment, the ligands within the meaning of the invention are in anionic form and can complex radioelements in cationic form, for example metal cations in the oxidation state (III). According to the present invention, the compounds of formula (I) are ligands.

The term “radioelement” is understood to mean any known radioisotope of a chemical element, whether natural or artificially produced. According to one embodiment, the radioelement is chosen from radioisotopes of yttrium, actinium, copper, gallium, indium, scandium and lanthanides. The term “lanthanides” denotes the atoms chosen from the group consisting of: La, Ce, Pr, Nd, Pm, Sm, Eu, Gd, Tb, Dy, Ho, Er, Tm, Yb and Lu.

The term “alkylphenyl” is understood to mean a linear or branched alkyl radical, preferably comprising from 1 to 20 carbon atoms, preferably from 1 to 8 carbon atoms, bonded to a phenyl. Preferably, the alkylphenyl is an octylphenyl radical.

The term “complex” is understood to mean the combination of a ligand as defined above with a chemical element, preferably a radioelement as defined above. The term “complex” is synonymous with “chelate”.

The term “thermodynamic stability” represents the affinity of the ligand for a given element, in particular a given metal. It concerns the equilibrium constant of the following reaction:

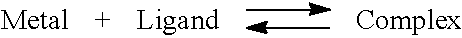

the mathematical expressions of which are as follows:

### Dissociation Constant

$$Kd = \frac{[ Metal ] \times [ Ligand ]}{[ Complex ]}$$

### Association Constant

$$Ka = \frac{[ Complex ]}{[ Metal ] \times [ Ligand ]}$$

The values are generally expressed in the form of decimal logarithm log Ka or −log of Kd. According to one embodiment, the complexes according to the invention are of high affinity. According to one embodiment, the complexes according to the invention have an equilibrium thermodynamic constant at least equal to 16 (Log Ka at least equal to 16).

The complexes formed according to the equilibrium reaction described above are liable to dissociate under the action of various factors (pH, presence of metals or competing ligands). This dissociation can have major consequences in the context of the use of the complexes in human medicine because it causes release of the metal into the body. In order to limit this risk, slow dissociation complexes are sought, that is to say complexes having good kinetic inertia. The kinetic inertia can be determined by dissociation tests in acidic medium. These experiments result in the determination, for each complex, of a half-life time (T_{1/2}) under defined conditions.

In the context of the invention, the term “to treat”, “treatment” or “therapeutic treatment” means reversing, relieving or inhibiting the progression of the disorder or ailment to which this term is applicable, or one or more symptoms of such a disorder.

The term “medical imaging” denotes the means for acquisition and reproduction of images of the human or animal body by means of various physical phenomena, such as the absorption or the emission of photons (visible, infrared, X-rays, gamma rays), nuclear magnetic resonance, the reflection of ultrasound waves, or radioactivity. According to one embodiment, the term “medical imaging” refers to X-ray imaging, MRI (magnetic resonance imaging), single-photon emission tomography (SPECT: single-photon emission computed tomography), positron emission tomoscintigraphy (PET) and luminescence. Preferably, the medical imaging method is X-ray imaging. SPECT if the complex according to the invention comprises a gamma emitter and PET if the complex according to the invention comprises a beta+ emitter.

The term “Lipiodol®” refers to an iodinated oil and preferentially to the pharmaceutical speciality Lipiodol®, an injectable solution manufactured and sold by Guerbet and consisting of ethyl esters of iodinated fatty acids of poppy oil. Lipiodol® is a product used in particular for visualization, localization and/or vectorization during transarterial chemoembolization of hepatocellular carcinoma at the intermediate stage, in adults, and also for the diagnosis by the selective hepatic arterial route of the hepatic extension of malignant or non-malignant hepatic lesions.

The term “organic acid” (or “organic acid function”) is understood to mean an organic compound (or an organic function) exhibiting acidic properties, that is to say capable of releasing an H⁺ or H₃O⁺ cation in an aqueous medium. Mention may be made, among organic acids, of carboxylic acids, sulfonic acids, phosphates and phosphonates. Preferably, the organic acid functions according to the invention are carboxyl groups. Such acid functions are salifiable and can exist in their basic form. In particular, these acid functions exist in the form of pharmaceutically acceptable salts, as defined below; for example, in the sodium or meglumine (1-deoxy-1-(methylamino)-D-glucitol or N-methyl-D-glucamine) salt form.

### The Iodinated Oils

The term “fatty acid” is understood to denote saturated or unsaturated aliphatic carboxylic acids exhibiting a carbon chain of at least 4 carbon atoms. Natural fatty acids have a carbon chain of 4 to 28 carbon atoms (generally an even number). The term “long-chain fatty acid” is used for a length of 14 to 22 carbons and “very-long-chain fatty acid” is used if there are more than 22 carbons. Conversely, the term “short-chain fatty acid” is used for a length of 4 to 10 carbons, in particular 6 to 10 carbon atoms, especially 8 or 10 carbon atoms. A person skilled in the art knows the associated nomenclature and in particular uses:
Cᵢ-Cₚ to denote a range of Cᵢ to Cₚ fatty acids,
Cᵢ+Cₚ, the total of the Cᵢ fatty acids and of the Cₚ fatty acids.
   For example:
the fatty acids of 14 to 18 carbon atoms are written as “C₁₄-C₁₈ fatty acids”,
the total of the C₁₆ fatty acids and of the C₁₈ fatty acids is written as C₁₆+C₁₈,
for a saturated fatty acid, a person skilled in the art will use the following nomenclature Ci:0, where i is the number of carbon atoms of the fatty acid. Palmitic acid, for example, will be denoted by the nomenclature (C16:0),
for an unsaturated fatty acid, a person skilled in the art will use the following nomenclature Ci:x n-N, where N will be the position of the double bond in the unsaturated fatty acid starting from the carbon opposite the acid group, i is the number of carbon atoms of the fatty acid and x is the number of double bonds (unsaturations) in this fatty acid. Oleic acid, for example, will be denoted by the nomenclature (C18:1 n-9).

Advantageously, the iodinated oil according to the invention comprises or consists of iodinated fatty acid derivatives, preferentially of ethyl esters of iodinated fatty acids, more preferentially of ethyl esters of iodinated fatty acids of poppy oil, of olive oil, of rapeseed oil, of peanut oil, of soybean oil or of walnut oil, more preferentially still of ethyl esters of iodinated fatty acids of poppy oil or of olive oil. More preferentially, the iodinated oil according to the invention comprises or consists of ethyl esters of iodinated fatty acids of poppy (also known as black poppy or Papaver somniferum var. nigrum) oil. Poppy oil, also known as poppyseed oil, preferentially contains more than 80% of unsaturated fatty acids (in particular linoleic acid (C18:2 n-6) and oleic acid (C18:1 n-9)), including at least 70% of linoleic acid and at least 10% of oleic acid. The iodinated oil is obtained from the total iodination of an oil, such as poppy oil, under conditions making possible bonding of one iodine atom for each double bond of the unsaturated fatty acids (Wolff et al., 2001, Medicine, 80, 20-36)), followed by a transesterification.

The iodinated oil according to the invention preferentially contains from 29% to 53% (w/w), more preferentially from 37% to 39% (w/w), of iodine.

Mention may be made, as examples of iodinated oils, of Lipiodol®, Brassiodol® (resulting from rapeseed (Brassica compestis) oil), Yodiol® (resulting from peanut oil), Oriodol® (resulting from poppy oil but in the form of fatty acid triglycerides) or Duroliopaque® (resulting from olive oil).

Preferentially, the iodinated oil is Lipiodol®, an iodinated oil used as contrast product and in certain interventional radiology procedures. This oil is a mixture of ethyl esters of iodinated and non-iodinated fatty acids of poppyseed oil. It consists predominantly (in particular more than 84%) of a mixture of ethyl esters of iodinated long-chain fatty acids (in particular C₁₈ fatty acids) resulting from poppyseed oil, preferentially of a mixture of ethyl monoiodostearate and of ethyl diiodostearate. The iodinated oil can also be an oil based on monoiodinated ethyl ester of stearic acid (C18:0) resulting from olive oil. A product of this type, called Duroliopaque®, was marketed a few years ago.

The main characteristics of Lipiodol® are as follows:

| Compounds | Proportions in the fatty acid mixture |
|---|---|
| Ethyl palmitate (Ethyl C16:0) | 4.6% to 6.7% (w/w), preferentially 4.8% (w/w) |
| Ethyl stearate (Ethyl C18:0) | 0.8% to 1.9% (w/w), preferentially 1.2% (w/w) |
| Ethyl monoiodostearate | 11.3% to 15.3% (w/w), preferentially 13.4% (w/w) |
| Ethyl diiodostearate | 73.5% to 82.8% (w/w), preferentially 78.5% (w/w) |

Other characteristics of Lipiodol ®:

| | |
|---|---|
| Iodine | 37% to 39% (w/w) (i.e. 480 mg/ml) |
| Viscosity | |
| at 37° C. | 25 mPa · s |
| at 20° C. | 50 mPa · s |
| Density | 1.268-1.290 g/cm³ at 20° C., preferentially 1.28 |

### Compounds of General Formula (I)

According to one embodiment, the compounds of general formula (I) are in salt form, preferably in the form of a pharmaceutically acceptable salt.

The term “pharmaceutically acceptable salt” denotes in particular salts which make it possible to retain the biological effectiveness and properties of the compounds according to the invention. Examples of pharmaceutically acceptable salts are found in Berge et al. ((1977), J. Pharm. Sd., Vol. 66, 1). For example, the compounds of general formula (I) are in the form of the sodium or meglumine (1-deoxy-1-(methylamino)-D-glucitol or N-methyl-D-glucamine) salt.

The invention also relates to solvates, such as hydrates, of the compounds of formula (I).

According to one embodiment, the compound of formula (I) is chosen from the group consisting of the following compounds:

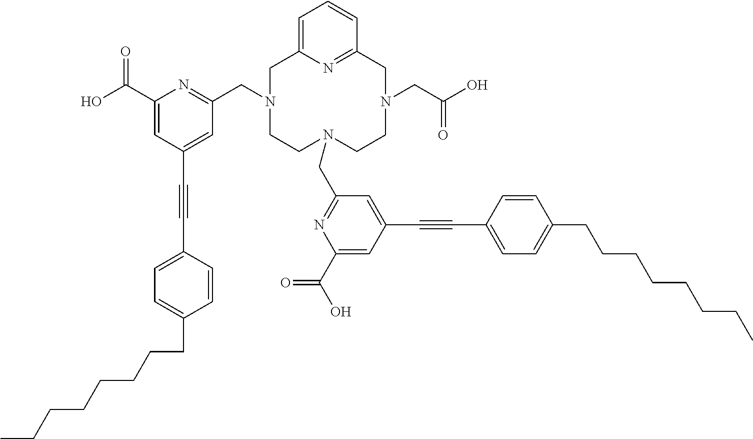 [SMILES: CCCCCCCCc1ccc(C#Cc2cc(CN3CCN(CC(=O)O)Cc4cccc(n4)CN(Cc4cc(C#Cc5ccc(CCCCCCCC)cc5)cc(C(=O)O)n4)CC3)nc(C(=O)O)c2)cc1]

(this compound is that of example 11a and has the nomenclature 6,6′-((9-(carboxymethyl)-3,6,9-triaza-1(2,6)-pyridinacyclodecaphane-3,6-diyl)bis(methylene))bis(4-((4-octylphenyl)ethynyl)picolinic acid))

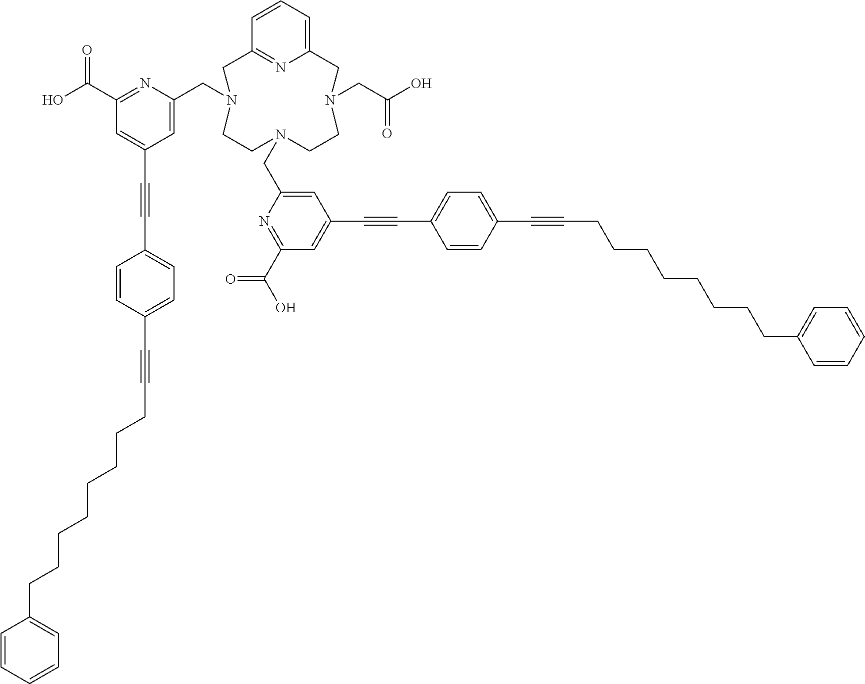 [SMILES: O=C(O)CN1CCN(Cc2cc(C#Cc3ccc(C#CCCCCCCCCc4ccccc4)cc3)cc(C(=O)O)n2)CCN(Cc2cc(C#Cc3ccc(C#CCCCCCCCCc4ccccc4)cc3)cc(C(=O)O)n2)Cc2cccc(n2)C1]

(this compound is that of example 11b and has the nomenclature 6,6′-((9-(carboxymethyl)-3,6,9-triaza-1(2,6)-pyridinacyclodecaphane-3,6-diyl)bis(methylene))bis(4-((4-(10-phenyldec-1-yn-1-yl)phenyl)ethynyl)picolinic acid))

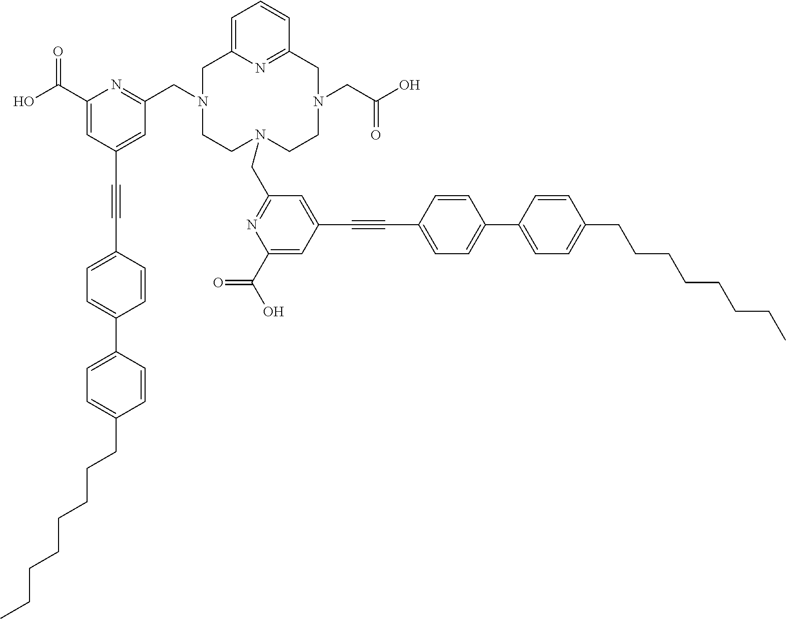 [SMILES: CCCCCCCCc1ccc(-c2ccc(C#Cc3cc(CN4CCN(CC(=O)O)Cc5cccc(n5)CN(Cc5cc(C#Cc6ccc(-c7ccc(CCCCCCCC)cc7)cc6)cc(C(=O)O)n5)CC4)nc(C(=O)O)c3)cc2)cc1]

(this compound is that of example 11c and has the nomenclature 6,6′-((9-(carboxymethyl)-3,6,9-triaza-1(2,6)-pyridinacyclodecaphane-3,6-diyl)bis(methylene))bis(4-((4′-octyl-[^{1,1}′-biphenyl]-4-yl)ethynyl)picolinic acid)) and one of their pharmaceutically acceptable salts.

According to a particular embodiment, the compound of formula (I) is the following compound:

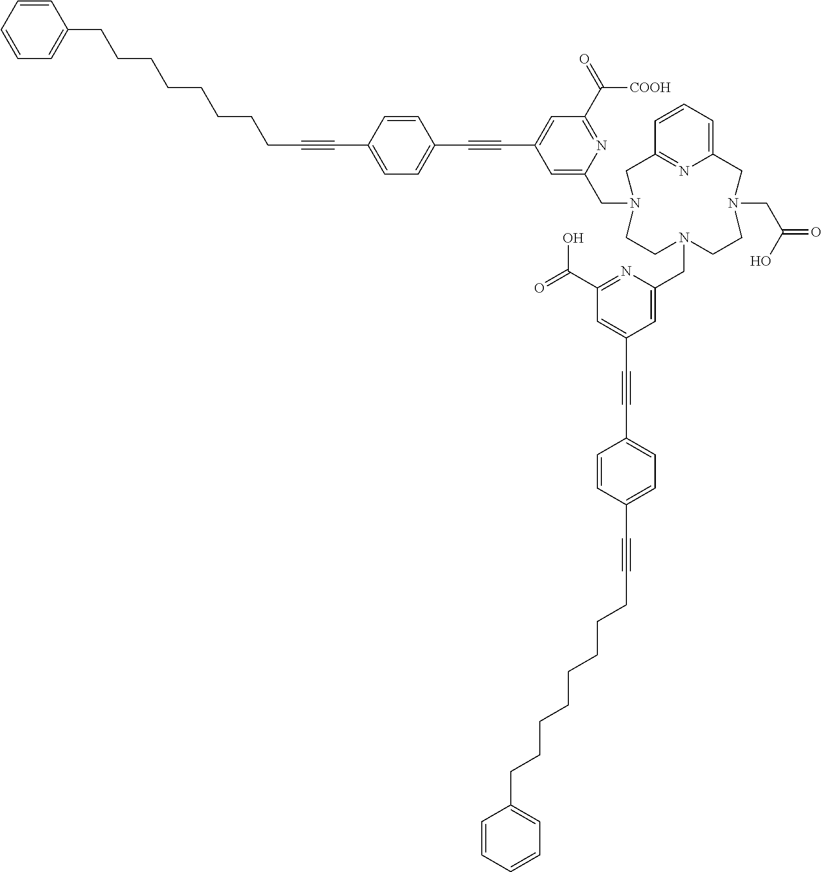 [SMILES: O=C(O)CN1CCN(Cc2cc(C#Cc3ccc(C#CCCCCCCCCc4ccccc4)cc3)cc(C(=O)O)n2)CCN(Cc2cc(C#Cc3ccc(C#CCCCCCCCCc4ccccc4)cc3)cc(C(=O)C(=O)O)n2)Cc2cccc(n2)C1]

or one of its pharmaceutically acceptable salts.

### Complexes

The invention also relates to a complex of a compound of formula (I) or of one of its pharmaceutically acceptable salts, as defined above, with a chemical element M, preferably a metal.

According to one embodiment, the compounds of general formula (I) are in the form of a neutral complex with the cations in the oxidation states III.

According to one embodiment, the chemical element M is a metal cation chosen from the group consisting of copper (II), gallium (III), indium (III), scandium (III), yttrium (III), samarium (III), terbium (III), holmium (III), lutetium (III), actinium (III) and manganese, preferably yttrium, lutetium, terbium, indium, gallium, copper and actinium. More preferentially still, the chemical element M is a metal cation chosen from the group consisting of yttrium (III), lutetium (III), copper (II) and actinium (III).
Preferably, M is a radioelement chosen from the radioactive isotopes of yttrium, lutetium, terbium, indium, gallium, copper, actinium and manganese.
According to a particular embodiment, the chemical element M is a radioelement chosen from the group consisting of ⁴⁴Sc(III), ⁴⁷Sc(III), ¹¹¹In(III), ¹⁵²Tb(III), ¹⁵⁵Tb(III), ¹⁴⁹Tb(III), ¹⁶¹Tb(III), ⁶⁴Cu(III), ⁶¹Cu(III), ⁶⁷Cu(II), ⁶⁸Ga(III), ⁹⁰Y(III), ¹⁵³Sm(III), ¹⁶⁶Ho(III), ¹⁷⁷Lu(III), ⁵²Mn and ²²⁵Ac(III), preferably ⁹⁰Y(III), ¹⁷⁷Lu(III), ⁶⁷Cu(II), ²²⁵Ac(III), ¹¹¹In(III), ¹⁵²Tb(III), ¹⁵⁵Tb(II), ¹⁴⁹Tb(III), ¹⁶¹Tb(III) and ⁶⁸Ga(III).

According to one embodiment, said complex is of following general formula (III):

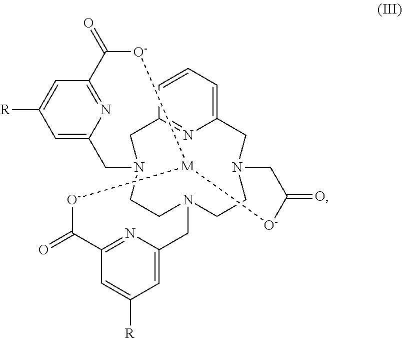

in which the groups R and M are as defined above.
The synthesis of such complexes is illustrated in examples 12 and 13.
According to a particular embodiment, the complex of formula (III) is chosen from the group consisting of the following complexes:

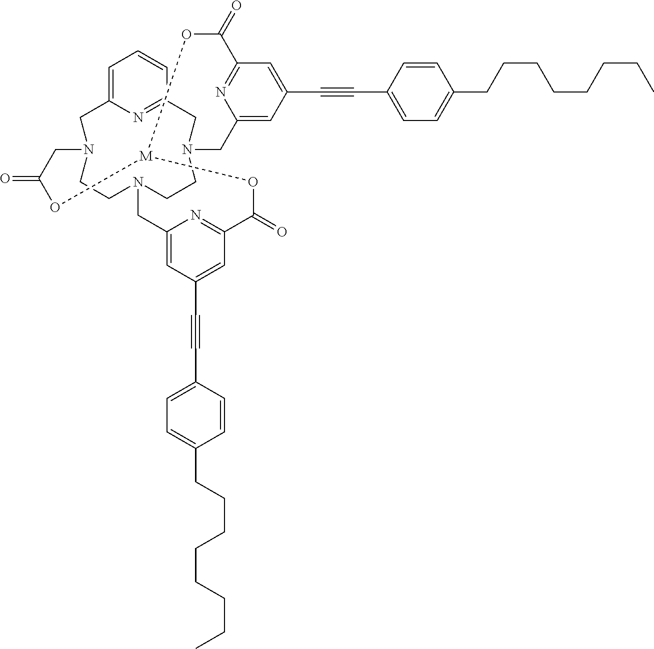

(this complex of formula (III) is formed with the compound of example 11a)

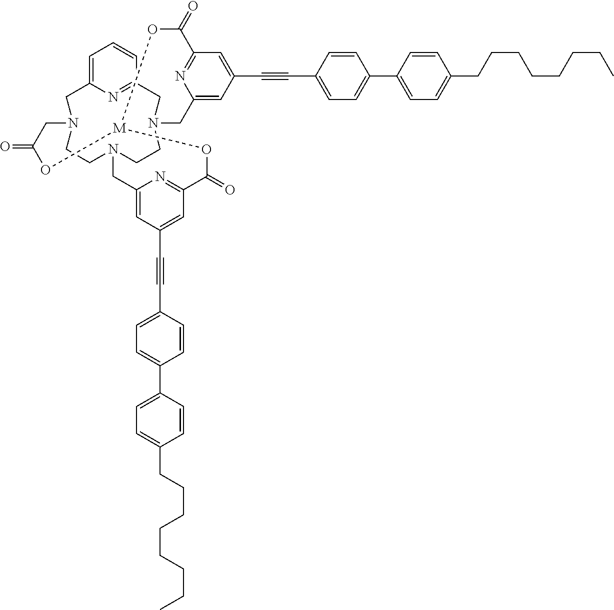

(this complex of formula (III) is formed with the compound of example 11c)
and

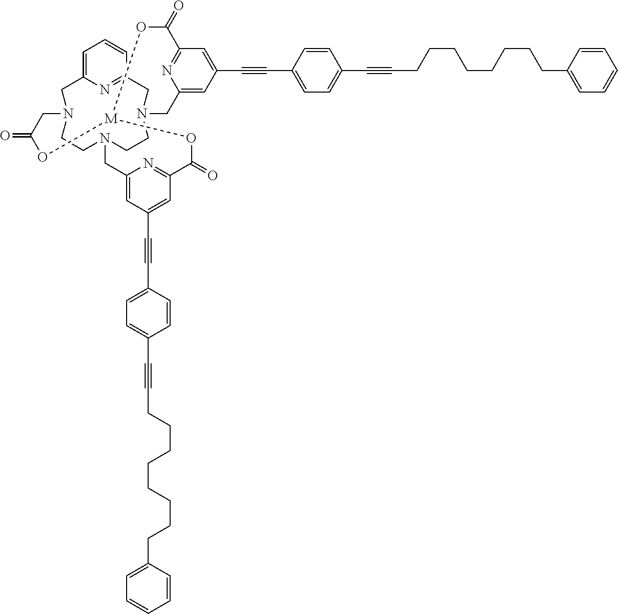

(this complex of formula (III) is formed with the compound of example 11b).

### Pharmaceutical Composition

The invention also relates to a pharmaceutical composition comprising a compound of formula (I) as defined above or a complex of formula (III) as defined above, and optionally one or more pharmaceutically acceptable excipient(s). The pharmaceutical composition can comprise a compound of formula (I) as defined above or a complex of formula (III) as defined above, in a pharmaceutically acceptable medium. By way of examples, mention may be made, as excipients, of radioprotectors or antioxidants. The term “radiolysis” is understood to mean a chemical reaction which is caused by ionizing radiation and which is liable to initiate or accelerate mechanisms of degradation of the radiopharmaceutical. A radioprotector has the property of blocking or limiting these radiochemical degradation phenomena.

The pharmaceutical composition can comprise an oily phase, in particular an iodinated oil. According to a particular embodiment, the pharmaceutical composition additionally comprises ethyl esters of iodinated fatty acids of poppy oil.

According to one embodiment, the pharmaceutical composition according to the invention comprises at least one pharmaceutically acceptable excipient. According to another embodiment, the pharmaceutical composition according to the invention does not comprise an excipient.

According to one embodiment, the pharmaceutical composition according to the invention consists of an iodinated oil and of compounds or of complexes according to the invention. Typically, the pharmaceutical composition according to the invention consists of Lipiodol® and of compounds or of complexes according to the invention. Lipiodol® consists of ethyl esters of iodinated fatty acids of poppy oil. Preferably, the pharmaceutical composition according to the invention consists of Lipiodol® and of the compound of example 11b, or else of Lipiodol® and of the complex of example 13a (i.e. yttrium-90 complex of the compound of example 11b) or 13c (i.e. lutetium-177 complex of the compound of example 11b).

Preferably, the pharmaceutical composition according to the invention is radioopaque and thus visible by X-ray radiography.

According to a particular embodiment, the pharmaceutical composition is an injectable composition. According to one embodiment, the pharmaceutical composition according to the invention is administered by intra-arterial hepatic injection.

The invention relates to a complex or to a pharmaceutical composition as are defined above, for its use in the treatment of cancers, in particular of cancers of the liver.

The invention also relates to a complex or to a pharmaceutical composition as are defined above, for its use in medical imaging.

The invention relates to the use of a complex as defined above for the preparation of a medicament for the treatment of cancers.

The invention also relates to the use of a complex or of a pharmaceutical composition as are defined above in medical imaging.

The invention relates to a method for the therapeutic treatment of a patient suffering from cancer, comprising the administration to said patient of a complex or of a pharmaceutical composition as are defined above. In particular, said treatment method does not comprise a stage of surgical treatment.

The invention also relates to a method for the medical imaging of a tumor, comprising:
a stage of administration to a patient suffering from cancer of a complex or of a pharmaceutical composition according to the invention; and
a stage of detection of the tumor by a medical imaging method.

The term “cancer” is understood to mean an abnormal cell proliferation (also known as tumor) within a normal tissue of the body. These cancer cells all derive from one and the same clone, a cell initiating the cancer, which has acquired certain characteristics enabling it to divide indefinitely. In the course of the development of the tumor, certain cancer cells may migrate from their site of production and form metastases.

Among cancers, mention may in particular be made of cancers of the liver, in particular primary liver cancers, preferably hepatocarcinomas. According to a particular embodiment, mention may be made, among cancers, of hepatocarcinoma, epithelioid hemangioendothelioma, cholangiocarcinoma, neuroendocrine tumors and metastases of other cancers, such as metastases of colorectal cancer.

According to a particular embodiment, the cancer is an intermediate-stage hepatocellular carcinoma, in adults.

### Process for the Preparation of the Compounds of General Formula (I) and Radiolabeling

The invention also relates to a preparation process for the compounds of general formula (I) according to the invention.

In this preparation process, the deprotection stages are known to a person skilled in the art and correspond to conventional reactions for hydrolysis of an amide. The functionalization stages are also known to a person skilled in the art and correspond to standard alkylation reactions (cf. Loic Bellouard, J. Chem. Soc., Perkin 1, (23), 1999, pages 3499-3505).

This preparation process is advantageously based on the reaction of an oxalic acid diester with pyclen, which makes it possible to block two nitrogen atoms of the pyclen (N-6 and N-9) in order to be able to act selectively on the third atom (N-3) which has been left free. After functionalization of the nitrogen in the 3-position, deprotection of the oxalamide group results in a pyclen which is substituted in the 3-position in a controlled manner, according to the following scheme 1:

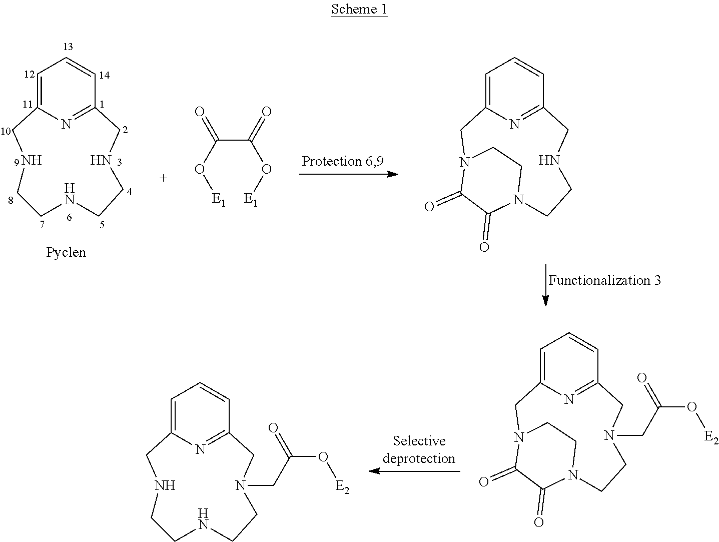

According to one embodiment, the protection stage is carried out in the presence of methanol.

The invention relates to a process for the preparation of the compounds of general formula (I) comprising a stage of functionalization of a compound of following general formula (IX):

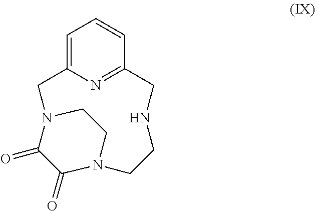

to form a compound of following general formula (X):

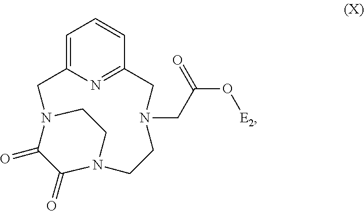

in which E₂ is a C₁-C₄ alkyl protecting group which can, for example, be chosen from the group consisting of methyl, ethyl, isopropyl and tert-butyl.

Said preparation process additionally comprises:
a stage of deprotection of the compound of general formula (X), in order to obtain a compound of following general formula (XI):

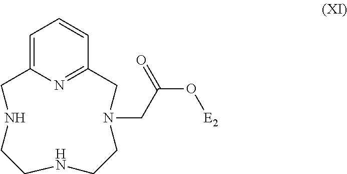

and
a stage of functionalization of the compound of general formula (XI) according to scheme 2, in order to obtain a compound of general formula (I) as defined above:

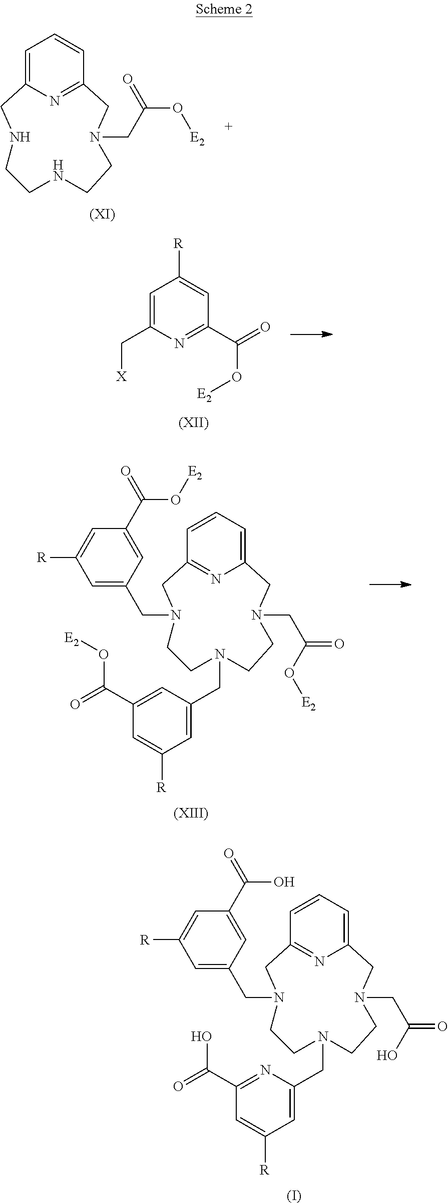

E₁, E₂ and E₃ being C₁-C₄ alkyl protecting groups which can, for example, be independently chosen from the group consisting of methyl, ethyl, isopropyl and tert-butyl.

The patent application also describes a compound of following general formula (X):

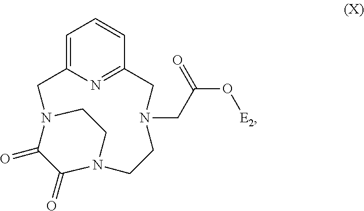

in which E₂ is a C₁-C₄ alkyl protecting group which can, for example, be chosen from the group consisting of methyl, ethyl, isopropyl and tert-butyl.

According to a particular embodiment of the process for the preparation of the compounds of formula (I), the preparation of a substituted picolinate intermediate is carried out via a derivative brominated in the 4-position, which makes it possible, by a palladium-catalyzed coupling reaction with an alkyne (“Sonogashira” reaction, Comprehensive Chirality, Volume 4, pages 18-32, 2012), to put in the chosen residue, according to scheme 3 below:

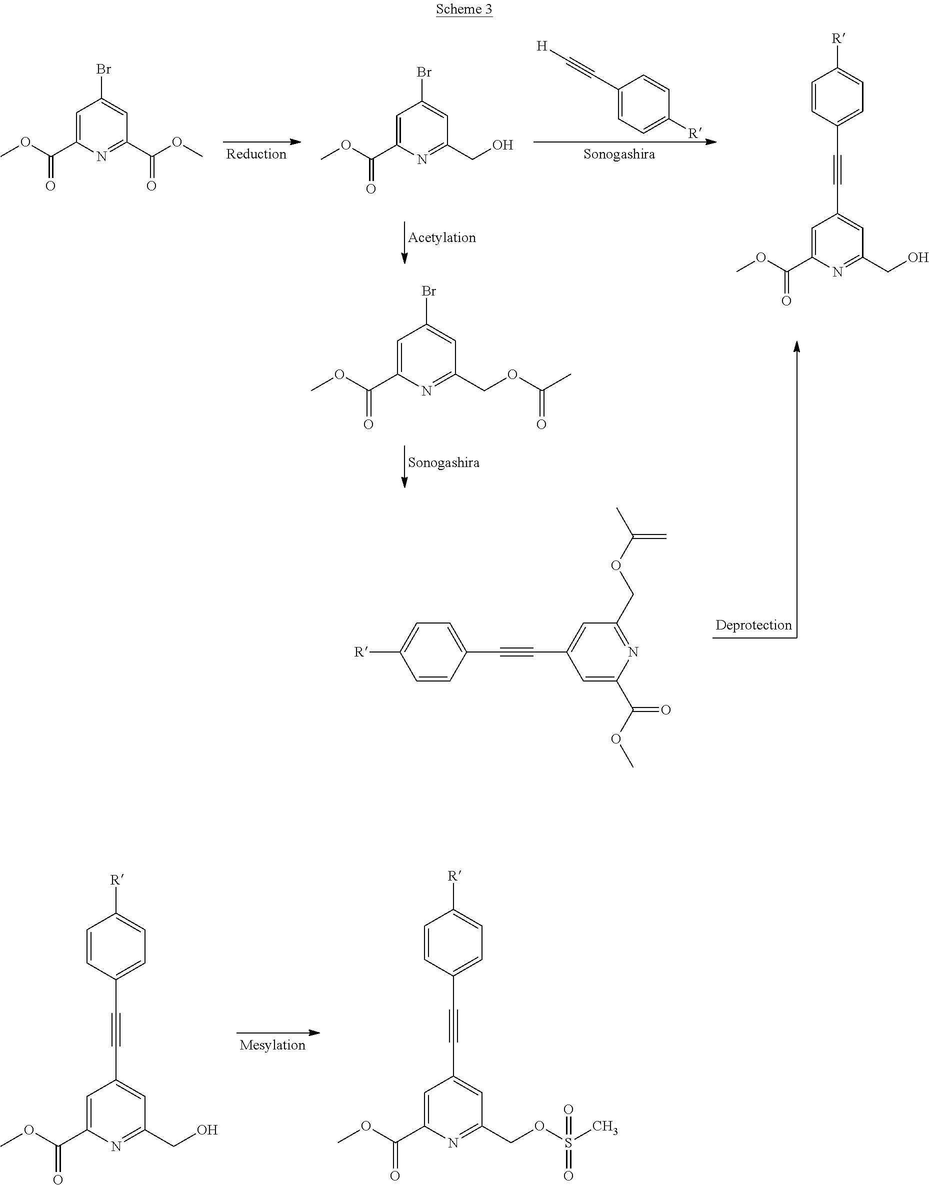

with R′ being a group of following formula-L1-(CH₂)ₙ-L2,
where
   L1 represents Ph or —C≡C— or CH₂,
   L2 represents H or Ph or alkylphenyl, and
   n of between 4 and 12.
      Preferably, R′ is one of the following radicals:

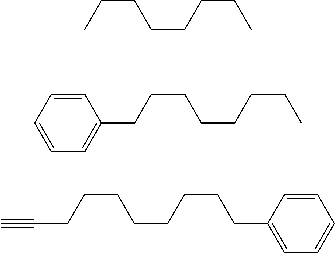

The invention also relates to a process for the radiolabeling of the compounds of general formula (I). Said radiolabeling process is preferably carried out at a pH of between 5 and 9, preferably between 5 and 7, preferably 5.2, in order to make possible complexing. According to a particular embodiment, said radiolabeling is carried out in the presence of acetate buffer in order to adjust the pH and thus make possible complexing. According to one embodiment, the radiolabeling is carried out in the presence of water or of alcohol, such as ethanol, or their mixtures.

The radiolabeling is carried out at a temperature of between 60° C. and 100° C., preferably between 80° C. and 100° C., more preferentially 80° C.

The radiolabeling results are presented in table 1 below. The radiochemical purity of the complex according to the invention after complexing with yttrium-90 is expressed as percentage of the total radioactivity engaged. The percentage of extraction of the radiolabeled complex into the Lipiodol® represents the fraction of the total radioactivity which is extracted into the oily phase.

TABLE 1

| | | | Lipiodol ® |
|---|---|---|---|
| | | | extraction |
| Ligand | ⁹⁰Y Complex | RCP % | % |
| Example 11a | Example 15 | 99.7 | 92.9 |
| Example 11c | Example 14 | 92.7 | 89.5 |
| Example 11b | Example 13a | 98.1 | 90.0 |

Ligand 1.10⁻³ M
RCP: Radiochemical purity

These data demonstrate the effectiveness of the radiolabeling of the ligands according to the invention and that the radiolabeled complexes according to the invention have a high affinity for the oily phase.
On conclusion of the radiolabeling operations, the radiolabeled complexes incorporated into the oily phase are engaged in a stability test in human serum. For this, the oily solutions are incubated at 37° C. with moderate stirring in the presence of human serum.
The distribution of the radioactivity in the oily phase and in the serum is measured at regular intervals in order to determine the fraction of radioactivity which diffuses toward the serum as a function of time. Extraction curves are thus drawn up and make it possible to evaluate the behavior of the different products and to compare them. These tests are described in example 16.

### DESCRIPTION OF THE FIGURES

FIG. 1: Release of yttrium-90 or indium-111 for the compounds of examples 13a and 13b respectively into the aqueous phase (physiological saline).

FIG. 2: Release of yttrium-90 into human serum (aqueous phase) for the compounds of examples 13a, 14 and 15.

FIG. 3: Release of yttrium-90 into human serum (aqueous phase) for the comparator compound 2.

FIG. 4: Release of yttrium-90 into human serum (aqueous phase) for the compounds according to the invention of examples 13a, 14 and 15 and for the comparator compounds 3 to 5.

FIG. 5: Evaluation of the ratio of the dose captured by the tumor compared to the healthy liver for the compound of example 13a.

FIG. 6: Results of biodistribution of the compound of example 13a compared to the compound of example 13b, Percentage of dose injected per organ.

FIG. 7: Release of lutetium-177 into human serum and into physiological saline for the compound according to the invention of example 13c.

The following examples are described by way of illustration of the present invention.

### EXAMPLES

### Equipment, Materials and Methods

### Radiolabeling of Ligands, Hot Synthesis of Complexes:

Yttrium-90 chloride is purchased from PerkinElmer Life Sciences and Indium-111 from Curium. Lutetium-177 nca (no carrier added) is supplied by ITM. The radioactivities brought into play in these examples are of between 28 μCi and 8.51 mCi for yttrium (1.04-314.87 MBq), 4.71 mCi (174 MBq) for indium and 673 MBq for lutetium.

The products (HPLC solvents, buffers, and the like) are used as is, without further purification. Unless otherwise specified, the ligand is dissolved in ethanol.

The experiments were carried out in crimped borosilicate glass vials. The vials were heated in a Bioblock heating block which makes it possible to heat up to 6 vials. When stirring was necessary, a Lab Dancer S40 (VWR) vortex device was used. The centrifugations were performed with an MF 20-R centrifuge (Awel).

The radioactivities were measured in a CRC-127R activimeter (Capintec), which was calibrated each morning.

The quality controls were carried out by thin layer chromatography (TLC) on Whatman 1 paper, with a 0.1% NEt₃/MeOH mixture as eluent. The radiochemical purities are determined using a Cyclone phosphor imager (PerkinElmer), using Optiquant software.

The high performance liquid chromatography (HPLC) analyses described according to method 10 are carried out on a Dionex Ultimate 3000 HPLC line equipped with a diode array detector and with a fLumo radiochromatographic detector (Berthold), managed by Chromeleon software.

In the synthesis methods described below, the commercial products and also the solvents originate essentially from the companies Sigma-Aldrich®, Merck and VWR®. The ambient temperature is of between 20° C. and 25° C. The evaporations of the solvents are carried out under reduced pressure, using a Buchi R-210 evaporator, at temperatures of approximately 40° C.

Purifications by flash chromatography are carried out using cartridges of irregular silica gels or neutral aluminum oxide of the Buchi brand (40 g, 80 g, 120 g, 220 g or 440 g) with the following devices:
CombiFlash NextGen 300+ from Teledyne ISCO® equipped with a 200 to 400 nm UV detector or a 200 to 800 nm UV-visible detector
Reveleris X2 from Buchi equipped with a UV or UV-visible detector (200 to 850 nm) and with an evaporative light scattering detector (ELSD).

Purifications on preparative HPLC columns are carried out on PuriFlash F4250 from Interchim® equipped with a 200 to 600 nm UV detector and with an ELSD according to method 11.

Analyses and reaction monitoring are carried out by TLC in a tank saturated with the vapors of the elution solvent. The support used is silica gel on a glass plate with a particle size of 60 Å with fluorescent indicator F254 or basic aluminum oxide on a glass plate or neutral aluminum oxide on an aluminum plate with a particle size of 60 Å with fluorescent indicator F254 from Merck®. The frontal ratio (Rf) of the compounds is defined by the following calculation:

$$Rf = \frac{Deposition line - compound distance}{Deposition line - solvent front distance}$$

Analyses and reaction monitoring are also carried out by high performance liquid chromatography on an Agilent line of the 1200 series equipped with a G1315D DAD SL UV or UV/visible detector and processed with EMPOWER® software or on a Shimadzu LCMS-2020 line equipped with an SPD-M30A UV or UV/visible detector and with a quadrupole mass spectrometer and then processed with Labsolution software. The sample introduced from the liquid chromatograph is subsequently sprayed and ionized under atmospheric pressure by electrospray (ESI) in positive (ES+) or negative (ES−) form. Infusions are also carried out on an Ultimate 3000 RS HPLC from ThermoFisher® with injections of 5 μl/min and mass detection by an amaZon X ion trap from Bruker®. The results are expressed by the mass/charge ratio (m/z).

### Methods of Analysis and of Reaction Monitoring

Different methods of analysis and of reaction monitoring were used for each of the compounds. They are described below and will be specified for each synthesis.

### Method 1: HPLC (High Performance Liquid Chromatography)

Instrument: Agilent HP1200; Column: Waters: Xbride amide 3.5 μm; 4.6×150 mm; Eluent A: Acetonitrile, Eluent B: Formate buffer 10 mM pH=3.3; Flow rate 1.0 ml/min; Temperature: 25° C.; Injection: 10 μl; Wavelength 254 nm; Gradient:

| Time | | |
|---|---|---|
| (minutes) | % A | % B |
| 0 | 95 | 5 |
| 15 | 60 | 40 |
| 18 | 95 | 5 |
| 20 | 95 | 5 |

### Method 2: LCMS (High Performance Liquid Chromatography/Mass Spectrometry)

Instrument: Shimadzu LC/MS; Column: Waters: Kinextex C8 100×2.1 mm 1.7 μm; Eluent A: 0.05% Trifluoroacetic acid (TFA)/water, Eluent B: Acetonitrile; Flow rate 0.5 ml/min; Temperature: 30° C.; Injection: 1 μl; Wavelength 210 nm; Gradient:

| Time | | |
|---|---|---|
| (minutes) | % A | % B |
| 0.01 | 50 | 50 |
| 5.00 | 5 | 95 |
| 10.00 | 5 | 95 |
| 10.01 | 50 | 50 |
| 15.00 | 50 | 50 |

### Method 3: LCMS

Instrument: Shimadzu LC/MS; Column: ThermoFisher: Hypersil Gold 50×2.1 mm 1.9 μm; Eluent A: 0.1% Formic acid/water (v/v), Eluent B: Acetonitrile; Flow rate 0.5 ml/min; Temperature: 60° C.; Injection: 1 μl; Wavelength 260 nm; Gradient:

| Time | | | Flow rate |
|---|---|---|---|
| in min | % Solution A | % Solution B | (ml/min) |
| 0 | 75 | 25 | 0.5 |
| 1 | 75 | 25 | 0.5 |
| 11 | 0 | 100 | 0.5 |
| 13 | 0 | 100 | 0.5 |
| 14 | 75 | 25 | 0.5 |
| 20 | 75 | 25 | 0.5 |

### Method 4: LCMS

Instrument: Shimadzu LC/MS; Column: ThermoFisher: Symmetry C18 150×4.6 mm 5 μm; Eluent A: 0.05% Trifluoroacetic acid/water (v/v), Eluent B: Acetonitrile; Flow rate 1 ml/min; Temperature: 25° C.; Injection: 1 μl; Wavelength 260 nm; Gradient:

| Time | | | Flow rate |
|---|---|---|---|
| in min | % Solution A | % Solution B | (ml/min) |
| 0 | 98 | 2 | 1 |
| 12 | 0 | 100 | 1 |
| 20 | 0 | 100 | 1 |
| 25 | 98 | 2 | 1 |
| 30 | 98 | 2 | 1 |

### Method 5: LCMS

Instrument: Shimadzu LC/MS; Column: Waters: Kinextex C8 100×2.1 mm 1.7 μm; Eluent A: 0.3% Formic acid/water (v/v), Eluent B: Acetonitrile; Flow rate 0.5 ml/min; Temperature: 30° C.; Injection: 1 μl; Wavelength 274 nm; Gradient:

| Time | | |
|---|---|---|
| (minutes) | % A | % B |
| 0.01 | 80 | 20 |
| 0.5 | 80 | 20 |
| 5 | 50 | 50 |
| 5.5 | 50 | 50 |
| 6.0 | 80 | 20 |
| 8.0 | 80 | 20 |

### Method 6: LCMS

Instrument: Shimadzu LC/MS; Column: Kinextex C8 100×2.1 mm 1.7 μm; Eluent A: 0.3% Formic acid/water (v/v), Eluent B: Acetonitrile; Flow rate 0.5 ml/min; Temperature: 30° C.; Injection: 1 μl; Wavelength 274 nm; Gradient:

| Time | | |
|---|---|---|
| (minutes) | % A | % B |
| 0.01 | 40 | 60 |
| 0.5 | 40 | 60 |
| 8.0 | 25 | 75 |
| 10.0 | 10 | 90 |
| 10.5 | 10 | 90 |
| 11.000 | 40 | 60 |
| 13.00 | 40 | 60 |

### Method 7: LCMS

Instrument: Shimadzu LC/MS; Column: Accucore C30 150×2.1 mm 2.6 μm; Eluent A: 0.3% Formic acid/water (v/v), Eluent B: Acetonitrile; Flow rate 0.6 ml/min; Temperature: 40° C.; Injection: 1 μl; Wavelength 320 nm; Gradient:

| Time in | | |
|---|---|---|
| minutes | % A | % B |
| 0.01 | 50 | 50 |
| 5.00 | 5 | 95 |
| 10.00 | 5 | 95 |
| 10.01 | 50 | 50 |
| 15.00 | 50 | 50 |

### Method 8: LCMS

Instrument: Shimadzu LC/MS; Column: Thermo Scientific Hypersil GOLD 150×3 mm 3 μm; Eluent A: 0.1% Formic acid/water (v/v); Eluent B: 0.1% Formic acid/acetonitrile (v/v); Flow rate 1 ml/min; Temperature: 60° C.; Injection: 1 μl; Wavelength 320 nm; Gradient:

| Time in | | |
|---|---|---|
| minutes | % A | % B |
| 0.01 | 50 | 50 |
| 11.00 | 0 | 100 |
| 13.00 | 0 | 100 |
| 16.00 | 50 | 50 |
| 20.00 | 50 | 50 |

### Method 9: LCMS

Instrument: Ultimate 3000 RS/amaZon X LC/MS; Column: Waters, Symmetry C18 50*2.1 mm 3.5 μm; Eluent A: 0.05% Trifluoroacetic acid/water (v/v); Eluent B: Acetonitrile; Flow rate 0.208 ml/min; Temperature: 60° C.; Injection: 1 μl; Gradient:

| Time in | | |
|---|---|---|
| minutes | % A | % B |
| 0.01 | 98 | 2 |
| 4.00 | 0 | 100 |
| 6.70 | 0 | 100 |
| 14.00 | 98 | 2 |
| 20.70 | 98 | 2 |

### Method 10: HPLC

Instrument: Dionex Ultimate 3000 HPLC; Column: ThermoFisher, Accucore C18 100×3 mm, 2.6 μm; Eluent A: Water; Eluent B: Acetonitrile; Flow rate 0.4 ml/min; Temperature: 25° C.; Gradient:

| Time in | | |
|---|---|---|
| minutes | % A | % B |
| 0 | 100 | 0 |
| 3.00 | 100 | 0 |
| 20.00 | 10 | 90 |
| 25.00 | 10 | 90 |
| 26.00 | 100 | 0 |
| 30.00 | 100 | 0 |

### Method 11: Prep HPLC (Purification on a Preparative Column)

Instrument: PuriFlash F4250; Column: Waters, Symmetry C18 150*30 mm 5 μm; Eluent A: 0.05% Trifluoroacetic acid/water (v/v); Eluent B: Acetonitrile; Flow rate 40 ml/min; Temperature: Ambient; Injection: 2 ml

### EXAMPLE 1: Synthesis of the monoalkylated macrocycle methyl 2-(3,6,9-triaza-1 (2,6)-pyridinacyclodecaphan-3-yl)acetate

### Example 1a: Synthesis of 5-aza-1(1,4)-piperazina-3(2,6)-pyridinacycloheptaphane-12,13-dione

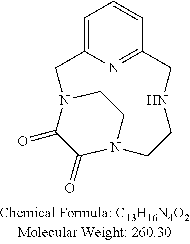 [SMILES: O=C1C(=O)N2CCN1CCNCc1cccc(n1)C2]

The pyclen base (Inorganic Chemistry, Volume 36, Issue 14, pages 2992-3000; 10 g, 0.047 mol) is dissolved in 400 ml of methanol and then a solution of diethyl oxalate (Sigma Aldrich, 1.01 equiv) dissolved in 200 ml of methanol is added with stirring under an inert atmosphere over 20 min. Stirring of the mixture is continued at ambient temperature for 3 hours. The solvent is subsequently evaporated under vacuum.

A white solid is obtained, w=12.7 g
Yield=quantitative
Support: basic aluminum oxide
Eluent: dichloromethane/methanol (9:1)
Rf=0.66
HPLC method: 1
RT (retention time)=9.0 min

### Example 1b: Synthesis of methyl 2-(1²,1³-dioxo-5-aza-1(1,4)-piperazina-3(2,6)-pyridina-cycloheptaphan-5-yl)acetate

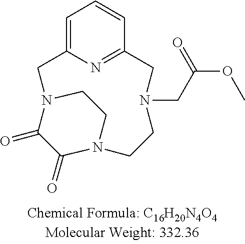 [SMILES: COC(=O)CN1CCN2CCN(Cc3cccc(n3)C1)C(=O)C2=O]

The intermediate obtained in example 1a (10.3 g, 0.040 mol) is suspended in 260 ml of acetonitrile and 1.55 equivalents of K₂CO₃ are added to the suspension. The mixture thus formed is stirred under an inert atmosphere for 15 minutes. 1.01 equiv. of methyl bromoacetate (Aldrich; reference: 147910-100G) dissolved in 260 ml of acetonitrile are added dropwise under an inert atmosphere over 30 minutes and the mixture is left stirring for 3 hours. The solvent is subsequently evaporated off under vacuum in order to obtain an oil.

The crude oil is dissolved in 970 ml of ethyl acetate and the salts are extracted with 35 ml of water. The organic phase is dried over Na₂SO₄ and filtered, and then the solvent is evaporated.

A white solid is obtained, w=13.5 g.
Yield=quantitative
Support: basic aluminum oxide
Eluent: dichloromethane/methanol (9:1)
Rf=0.85
HPLC method: method 1
RT=2.7 min

### Example 1b′: Synthesis of t-butyl 2-(1²,1³-dioxo-5-aza-1(1,4)-piperazina-3(2,6)-pyridina-cycloheptaphan-5-yl acetate

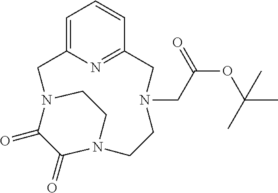 [SMILES: CC(C)(C)OC(=O)CN1CCN2CCN(Cc3cccc(n3)C1)C(=O)C2=O]

The synthesis is carried out according to the procedure described in example 1b, using t-butyl bromacetate (124230-10G, Aldrich) instead of methyl bromoacetate in the process described in example 1b.

A yellow oil is thus obtained.
w=710 mg.
Yield=99%.

### Example 1c: Synthesis of methyl 2-(3,6,9-triaza-1(2,6)-pyridinacyclodecaphan-3-yl)acetate

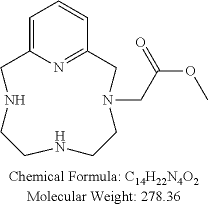 [SMILES: COC(=O)CN1CCNCCNCc2cccc(n2)C1]

A solution of 13.3 g of the intermediate described in example 1b and 11 ml of 98% sulfuric acid diluted in 265 ml of methanol is heated at reflux for 18 h. The solution is subsequently cooled to ambient temperature before adding 138 ml of Amberlyst® A21 resin (Aldrich; reference 216410-1KG) and is then left stirring for 30 minutes. The solution is filtered and the resin is rinsed with methanol.

The oil is washed three times with 50 ml of ethyl ether in order to remove the dimethyl oxalate.

The oil is dissolved in dichloromethane, the solution is dried over MgSO₄ and then filtered, and the solvent is evaporated.

The crude product is purified by flash chromatography using a 440 g neutral aluminum oxide cartridge with a dichloromethane/methanol gradient.

A white solid is obtained, w=4.81 g
Yield=43%
Support: neutral aluminum oxide
Eluent: dichloromethane/methanol (9:1)
Rf=0.46
HPLC method: 1
RT=6.6 min.

The following examples 2, 3 and 4 present the synthesis of the alkynes.

### EXAMPLE 2: Synthesis of 1-ethynyl-4-(10-phenyldec-1-yn-1-yl)benzene

### Example 2a: Synthesis of dec-9-yn-1-ylbenzene

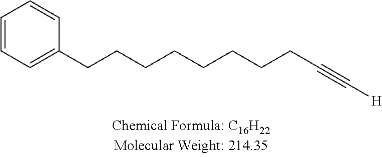 [SMILES: C#CCCCCCCCCc1ccccc1]

The lithium acetylide, ethylenediamine complex (Aldrich, reference 186155, 1444 mg, 2 equiv.) is diluted in 66 ml of a pentane/DMSO (7:3) mixture. The solution is stirred and degassed twice with nitrogen and then cooled to 0° C. 1-Bromo-8-phenyloctane (Interchim, purity 95%; reference OR8184; 2000 mg; 7.06 mmol) dissolved in 7 ml of the DMSO/Et₂O (1:1) mixture is added and then the reaction medium is stirred vigorously at ambient temperature for 22 hours. The solution is cooled to 0° C. and then 160 ml of saturated NH₄Cl solution are carefully added. The product is extracted with 2×160 ml of diethyl ether, dried over sodium sulfate (Na₂SO₄), filtered and concentrated to dryness. The crude yellow oil is purified by flash chromatography with a silica cartridge (40 g, Buchi; reference 14000024) with a heptane/dichloromethane gradient.

A colorless liquid is obtained
Yield=90%
Support: silica
Eluent: heptane
Rf=0.34
LCMS method: 2
RT=3.9 min

¹H NMR (300 MHZ, CDCl₃): δ 7.29 (m, 2H, phenyl), 7.19 (m, 3H, phenyl), 2.62 (t, J=7.5 Hz, 2H, R—CH₂-phenyl), 2.20 (td, J=6.9 Hz and J=2.6 Hz, 2H, R—CH₂-alkyne), 1.95 (t, J=2.7 Hz, 1H, —C≡CH), 1.67-1.35 (m, 12H, lipophilic chain).

### Example 2b: Synthesis of trimethyl((4-(10-phenyldec-1-yn-1-yl)phenyl)ethynyl)silane

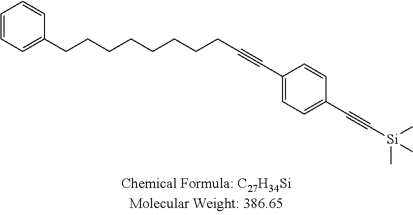 [SMILES: C[Si](C)(C)C#Cc1ccc(C#CCCCCCCCCc2ccccc2)cc1]

((4-Bromophenyl)ethynyl)trimethylsilane (2000 mg, 7.90 mmol; Aldrich, reference 494011) is diluted in 19 ml of diisopropylamine, then 1,1′-bis(diphenylphosphino) ferrocene (0.02 equivalent; Aldrich, reference 697230), copper iodide (0.06 equivalent; Aldrich, reference 03140) and triphenylphosphine (0.04 equivalent; Aldrich, reference T84409) are added. The solution is degassed under an inert atmosphere and is then heated to 90° C. The alkyne obtained in example 2a (1.1 equivalent) is added through a septum and then the reaction medium is stirred under hot conditions for 19 hours. The solution is cooled to ambient temperature and filtered through glass paper, and the residues are rinsed with diethyl ether. The filtrate is concentrated to dryness under reduced pressure. The residue is taken up in diethyl ether, washed with a saturated NaCl solution, dried over MgSO₄, filtered and concentrated to dryness. The black liquid is adsorbed on silica gel 60A (Merck; reference: 1.09385.2500) and purified on a silica cartridge (40 g; Buchi, reference 14000024), elution being carried out with a heptane/dichloromethane mixture.

A colorless liquid of weight w=1418 mg is obtained
Yield=93%
Support: silica
Eluent: heptane/dichloromethane (80:20)
Rf=0.27
LCMS method: 2
RT=5.9 min

¹H NMR (60 MHz, CDCl₃): δ 7.31 and 7.18 (m, 9H, phenyl), 2.62 and 2.26 (m, 4H, R—CH₂-phenyl and R—CH₂-alkyne), 1.33 (m, 12H, 6 CH₂ alkyl chain), 0.22 (s, 9H, 3×CH₃ of TMS).

### Example 2c: 1-Ethynyl-4-(10-phenyldec-1-yn-1-yl)benzene

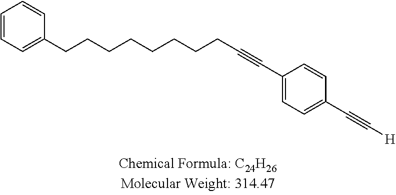 [SMILES: C#Cc1ccc(C#CCCCCCCCCc2ccccc2)cc1]

The intermediate obtained in example 2b (1418 mg, 3.67 mmol) is diluted with 2.2 ml of anhydrous tetrahydrofuran. The solution is cooled in a water/ice bath and then 4.4 ml (1.2 equiv.) of 1M tetrabutylammonium fluoride in THF (Aldrich, reference 216143) are added dropwise through a septum with a syringe and a needle. The reaction medium is stirred at ambient temperature for 2 hours. 12 ml of water are then added before carrying out 3 ex-tractions with 20 ml of diethyl ether in order to extract the product. The organic phases are combined, dried over Na₂SO₄, filtered and concentrated to dryness. The yellow liquid thus obtained is purified on a silica cartridge (40 g, Buchi, reference 14000024), elution being carried out with heptane.

A colorless liquid of weight w=773 mg is obtained
67%
Support: silica
Eluent: heptane/dichloromethane (80:20)
Rf=0.43
LCMS method: 2
RT=4.9 min

¹H NMR (60 MHZ, CDCl₃): δ 7.47-7.18 (m, 9H, phenyl), 3.08 (s, 1H, CH alkyne), 2.70-2.27 (m, 4H, R—CH₂-phenyl and R—CH₂-alkyne), 1.33 (m, 12H, 6×CH₂ alkyl chain).

### EXAMPLE 3: Synthesis of 1-ethynyl-4-octylbenzene

### Example 3a: Synthesis of trimethyl((4-octylphenyl)ethynyl)silane

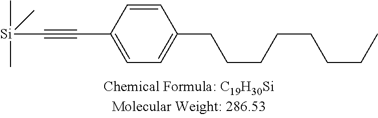 [SMILES: CCCCCCCCc1ccc(C#C[Si](C)(C)C)cc1]

1-Bromo-4-(n-octyl)benzene (3000 mg, 11.14 mmol, 1 equiv.; Alfa Aesar, reference A14676.06) is diluted in 27 ml of diisopropylamine, then 0.02 equivalent of 1,1′-bis(diphenylphosphino) ferrocene (Aldrich, reference 697230), 0.06 equivalent of copper iodide (Aldrich, reference 03140) and 0.04 equivalent of triphenylphosphine (Aldrich, reference T84409) are added. The solution is degassed under an inert atmosphere and is then heated to 85° C. before adding trimethylsilylethyne (1.1 equiv; Aldrich, reference 218170) thereto through a septum. The reaction medium is heated for 19 hours, then cooled to ambient temperature and filtered through glass paper, and the salts are rinsed with diethyl ether. The filtrate is concentrated to dryness under reduced pressure. The residue is taken up in diethyl ether and then washed with a saturated NaCl solution. The organic phase is subsequently dried over MgSO₄ and filtered, and the solvent is evaporated. A black liquid is obtained which will be adsorbed on silica gel 60 (Merck, reference 1.09385.2500) and purified on an 80 g silica cartridge with a heptane/dichloromethane eluent gradient.

A yellow liquid is obtained, w=2371 mg
Yield=74%
Support: silica
Eluent: heptane
Rf=0.43
LCMS Method: method 3
RT=9.9 min

### IR:

| | Type of | Specific | Absorption | |
|---|---|---|---|---|
| Bond | bond | type of bond | peak cm⁻¹ | Appearance |
| ν C—C | C≡C | Disubstituted | 2157 cm⁻¹ | Weak peak |
| | | alkyne | | |

### Example 3b: Synthesis of 1-ethynyl-4-octylbenzene

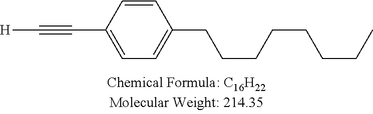 [SMILES: C#Cc1ccc(CCCCCCCC)cc1]

The intermediate obtained in example 3a (2254 mg, 7.87 mmol) is diluted in 11.8 ml of anhydrous THF and the medium is then conditioned with nitrogen. The solution is cooled in a water/ice bath and then 9.4 ml (1.2 equivalents) of 1M tetrabutylammonium fluoride in THF (Aldrich, reference 216143) are added dropwise through a septum. The reaction medium is stirred at ambient temperature for 2 hours. At the end of the reaction, 40 ml of water are added. The product is extracted with 80 ml of diethyl ether. The organic phase is dried over Na₂SO₄ and filtered, and the solvent is evaporated. A crude yellow liquid is obtained which will be purified with a silica cartridge (80 g, Buchi, reference 140000025) with the eluent heptane.

A colorless liquid is obtained, w=1162 mg
Yield=68%
Support: silica
Eluent: heptane
Rf=0.42
LCMS method: 3
RT=8.2 min

### Infrared:

| | | Specific type | Absorption | |
|---|---|---|---|---|
| Bond | Type of bond | of bond | peak cm⁻¹ | Appearance |
| ν C—H | C≡C | True alkyne | 3297 cm⁻¹ | Moderate peak |

### EXAMPLE 4: Synthesis of 4-ethynyl-4′-octyl-1,1′-biphenyl

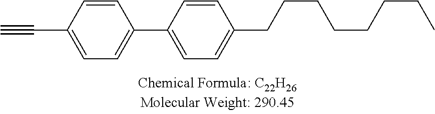 [SMILES: C#Cc1ccc(-c2ccc(CCCCCCCC)cc2)cc1]

1-Bromo-4-(n-octyl)benzene (592 mg, 2.2 mmol; Alfa Aesar, reference A14676.06), 4-((tri-methylsilyl)ethynyl)benzeneboronic acid pinacol ester (1321 mg, 2 equiv.; Interchim, H51697) and Cs₂CO₃ (3.48 eq.; Alfa Aesar, reference 10924) are diluted in 10 ml of tetrahydrofuran and 4 ml of water and then the solution is purged with nitrogen. Palladium (II) acetate (0.04 equiv., Aldrich reference: 520764) and triphenylphosphine (0.02 equiv.) are added and then the reaction medium is heated at 70° C. for 20 hours with the exclusion of light. The solution is cooled to ambient temperature and extracted with diethyl ether, then the organic phase is dried over Na₂SO₄ and filtered, and the solvent is evaporated. The black oil is purified by flash chromatography on a silica cartridge (40 g, Buchi; reference: 140000024) with the eluent heptane/AcOEt. A white/yellow solid is obtained after evaporation.

The purified intermediate obtained (980 mg, 2.70 mmol) is diluted in 2.70 ml of anhydrous THF and then the medium is conditioned with nitrogen. The solution is cooled in a water/ice bath and then 4.59 ml (1.7 eq.) of 1M TBAF in THF are added dropwise through a septum. The reaction medium is stirred at ambient temperature for 2 hours. At the end of the reaction, 10 ml of water are added. The product is extracted with 40 ml of diethyl ether. The organic phase is dried over Na₂SO₄ and filtered, and the solvent is evaporated. A solid is obtained which will be purified with a silica cartridge (40 g, Buchi) with the eluent heptane/ethyl acetate.

A white/yellow solid is obtained, w=784 mg
Yield=55%

| | | Specific type | Absorption | |
|---|---|---|---|---|
| Bond | Type of bond | of bond | peak cm⁻¹ | Appearance |
| ν C—H | C≡C | True alkyne | 3306 cm⁻¹ | Moderate peak |

The following examples 5, 6, 7 and 8b illustrate the Sonogashira reaction and the synthesis of lipophilic picolinates.

### EXAMPLE 5: Methyl 6-(hydroxymethyl)-4-((4-(10-phenyldec-1-yn-1-yl)phenyl)ethynyl)picolinate

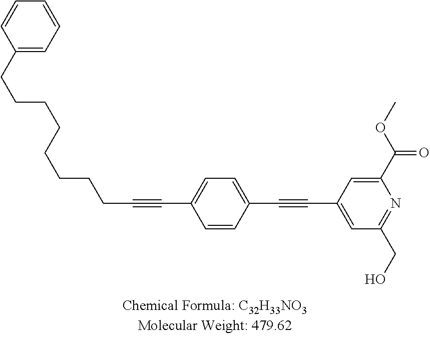 [SMILES: COC(=O)c1cc(C#Cc2ccc(C#CCCCCCCCCc3ccccc3)cc2)cc(CO)n1]

Methyl 4-bromo-6-(hydroxymethyl)picolinate (890 mg, 3.621 mmol; 1 equiv.; Interchim, reference 20210326; synthesis described in the patent WO 2017/109217, page 44) is diluted in 22 ml of dimethylformamide. The solution is conditioned under an inert atmosphere and then 7 ml of triethylamine, Pd(PPh₃)₂Cl₂ (0.05 equiv.), PPh₃ (0.1 equiv.) and CuI (0.1 equiv.) are added. After stirring for a few minutes, the alkyne obtained in example 2c (1-ethynyl-4-(10-phenyldec-1-yn-1-yl)benzene) (1.2 equiv.) is added to the reaction medium and the solution is heated to 110° C. The solution is cooled to ambient temperature and then 100 ml of diethyl ether are added. The organic phase is washed with 50 ml of saturated NH₄Cl solution and then with 50 ml of saturated NaCl solution, and then it is dried with Na₂SO₄, filtered and concentrated to dryness. A black oil is obtained which will be adsorbed on silica gel 60A and purified by flash chromatography with an 80 g silica cartridge and the heptane/AcOEt mixture.

A beige solid is obtained, w=923 mg
Yield=53%
TLC: silica
Eluent: heptane/AcOEt (4:6)
Rf=0.3
HPLC: method 4
RT=16.4 min m/z (ES+)=480.31

NMR: ¹H NMR (60 MHZ, CDCl₃): δ 8.19 and 7.72 (m, 2H, pyridine), 7.67-7.31 (m, 9H, phenyl), 4.97 (s, 2H, CH₂OH), 4.10 (s, 3H, CH₃ ester), 3.47 (m, 1H, OH primary alcohol), 2.82-2.43 (m, 4H, R—CH₂-phenyl and R—CH₂-alkyne), 1.47 (m, 12H, 6 CH₂ alkyl chain).

### EXAMPLE 6: Synthesis of methyl 6-(hydroxymethyl)-4-((4-octylphenyl)ethynyl)picolinate

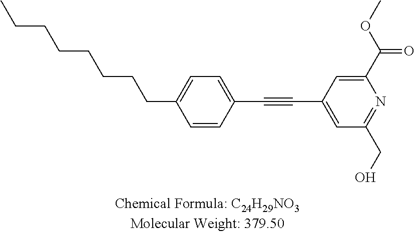 [SMILES: CCCCCCCCc1ccc(C#Cc2cc(CO)nc(C(=O)OC)c2)cc1]

Methyl 4-bromo-6-(hydroxymethyl)picolinate (1500 mg, 6.10 mmol; 1 equiv.; Interchim, reference 20210326; synthesis described in the patent application WO 2017/109217 on page 44) is diluted in 37 ml of anhydrous tetrahydrofuran. Then two degassing operations with nitrogen are carried out successively before adding 12 ml of triethylamine, Pd(PPh₃)₂Cl₂ (0.05 equiv.), PPh₃ (0.1 equiv.) and CuI (0.1 equiv.). After stirring for a few minutes, the alkyne obtained in example 3b, (1-ethynyl-4-octylbenzene) (1.2 equiv.), is added to the reaction medium and the solution is heated to 40° C. The solution is cooled to ambient temperature and filtered through glass paper (Whatman), and the residues are rinsed with 100 ml of Et₂O. The filtrate is washed with 100 ml of saturated NH₄Cl solution and 40 ml of saturated NaCl solution, and then the organic phase is dried over Na₂SO₄, filtered and concentrated to dryness. A black oil is obtained which will be adsorbed on silica gel 60A and purified by flash chromatography with a silica cartridge (Buchi; 80 g) and the heptane/AcOEt mixture. A white solid is obtained, w=1852 mg
Yield=80%
TLC: silica Eluent: heptane/AcOEt (8:2)
Rf=0.45
HPLC: method 4
RT=15.3 min
m/z (ES+)=380.28

### EXAMPLE 7: Synthesis of methyl 6-(hydroxymethyl)-4-((4′-octyl-[^{1,1}′-biphenyl]-4-yl)ethynyl)picolinate

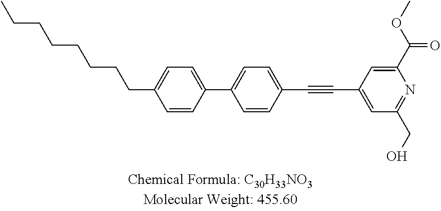 [SMILES: CCCCCCCCc1ccc(-c2ccc(C#Cc3cc(CO)nc(C(=O)OC)c3)cc2)cc1]

Methyl 4-bromo-6-(hydroxymethyl)picolinate (4.88 mmol; 1 equiv.; Interchim, reference 20210326; synthesis described in the patent application WO 2017/109217 on page 44), anhydrous THF (6.1 ml/mmol) and, after 2 degassing operations with nitrogen, the alkyne obtained in example 4 (4-ethynyl-4′-octyl-1,1′-biphenyl) (1.1 equiv.), Et₃N (2 ml/mmol), Pd(PPh₃)₂Cl₂ (0.1 equiv.) and CuI (0.1 equiv.) are mixed. The solution becomes black and the reaction medium is stirred at 40° C. in an inert environment. The solution is cooled to ambient temperature and filtered on glass paper (Whatman), and the residues are rinsed with 100 ml of Et₂O. The organic phase is washed twice with 100 ml of saturated NH₄Cl solution and 1×100 ml of saturated NaCl solution. It is subsequently dried over Na₂SO₄, filtered and concentrated to dryness. A black oil is obtained which will be adsorbed on silica and purified by flash chromatography with a silica cartridge and the heptane/AcOEt mixture.

A beige solid is obtained, w=1668 mg
Yield=75%
m/z (ES+)=456

Example 8 below presents the synthesis of the compound obtained in example 5, (methyl 6-(hydroxymethyl)-4-((4-(10-phenyldec-1-yn-1-yl)phenyl)ethynyl)picolinate), via a reaction for the protection of the alcohol by acetylation.

### Example 8

### Example 8a: Synthesis of methyl 6-(acetoxymethyl)-4-bromopicolinate

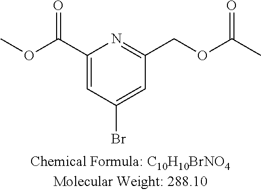 [SMILES: COC(=O)c1cc(Br)cc(COC(C)=O)n1]

The intermediate methyl 6-(hydroxymethyl)-4-bromopicolinate (25.409 g; 103.26 mmol) is diluted in 516 ml of dichloromethane and then the medium is conditioned under a nitrogen atmosphere. 26 ml of triethylamine are added in one go, acetic anhydride (7.6 equiv.; Aldrich, 242845) is added dropwise via a dropping funnel and then the reaction medium is stirred at ambient temperature for 2 hours. The organic phase is washed with 50 ml of de-ionized water, dried over Na₂SO₄, filtered and concentrated to dryness.

A white solid is obtained, w=33.65 g.
Yield=quantitative
Support: Silica gel
Eluent: heptane/AcOEt (4:6)
Rf=0.6
HPLC method: 5 RT=3.56 min
m/z (ES+)=288

NMR: ¹H NMR (60 MHZ, CDCl₃): δ 8.19 and 7.72 (m, 2H, pyridine), 5.35 (s, 2H, CH₂Ac), 4.07 (s, 3H, CH₃ ester), 2.25 (s, 3H, CH₃ acetate)

### Example 8b: Synthesis of methyl 6-(acetoxymethyl)-4-((4-(10-phenyldec-1-yn-1-yl)phenyl)ethynyl)picolinate

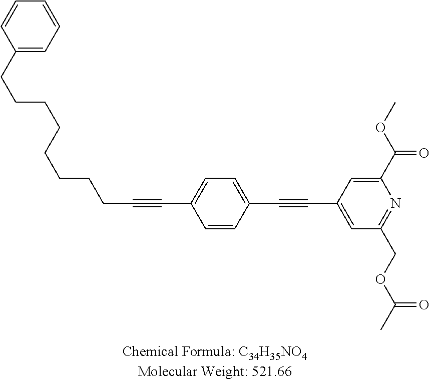 [SMILES: COC(=O)c1cc(C#Cc2ccc(C#CCCCCCCCCc3ccccc3)cc2)cc(COC(C)=O)n1]

The compound obtained in example 8a (20.53 g, 71.36 mmol) is diluted in 800 ml of tetrahydrofuran and the solution is then conditioned under an inert atmosphere. Triphenylphosphine (0.1 equiv.), Pd(PPh₃)₂Cl₂ (0.05 equiv.), CuI (0.1 equiv.) and 143 ml of Et₃N are added to the solution. The compound obtained in example 2c, in solution in 200 ml of tetrahydrofuran, is added to the reaction medium in one go and then the solution is heated at 60° C. for 50 min. The reaction medium is cooled to ambient temperature, filtered and rinsed with 250 ml of tetrahydrofuran. The solvent is evaporated under vacuum. The crude product is then redissolved in 910 ml of diethyl ether and filtered, and the organic phase is then washed with 500 ml of saturated NH₄Cl solution, 500 ml of saturated Na₂CO₃ solution and 250 ml of saturated NaCl solution. The organic phase is dried over Na₂SO₄, filtered and then concentrated under vacuum. A brown solid is obtained which is recrystallized in a heptane/AcOEt mixture with hot filtration.

The solid is subsequently rinsed with 100 ml of cold AcOEt in order to obtain a white solid, w=35.58 g
Yield=96%
Support: silica gel
Eluent: heptane/AcOEt (4:6)
Rf=0.65
HPLC method: RT=8.15 min
m/z (ES+)=522

NMR: ¹H NMR (60 MHZ, CDCl₃): δ 8.11 and 7.57 (m, 2H, pyridine), 7.42-7.19 (m, 9H, phenyl), 5.30 (s, 2H, CH₂Ac), 3.99 (s, 3H, CH₃ ester), 2.59-2.30 (m, 4H, R—CH₂-phenyl and R—CH₂-alkyne), 2.17 (s, 3H, CH₃ acetate), 1,35 (m, 12H, 6 CH₂ alkyl chain).

### Example 8c: Synthesis of the Compound According to Example 5 (methyl 6-(hydroxymethyl)-4-((4-(10-phenyldec-1-yn-1-yl)phenyl)ethynyl)picolinate) from the Intermediate Obtained in Example 8b

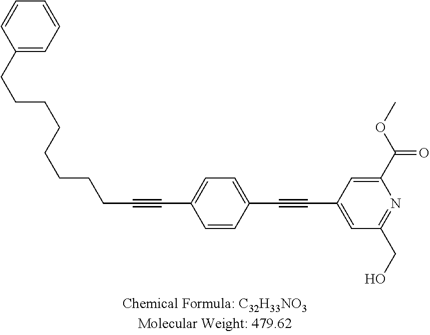 [SMILES: COC(=O)c1cc(C#Cc2ccc(C#CCCCCCCCCc3ccccc3)cc2)cc(CO)n1]

The intermediate obtained in example 8b (33.36 g, 63.95 mmol) is diluted in 255 ml of methanol. 27 ml of triethylamine (3 equiv.) are added and then the reaction medium is heated at reflux for 23 hours. The reaction medium is filtered and then the filtrate is cooled in an acetone/dry ice bath. After filtration, a white solid is obtained of weight w=22.91 g.
Yield=75%

The following example 9 illustrates the reactions for the activation of the alcohol in the mesylate form.

### EXAMPLE 9: Synthesis of Substituted methyl-4-6-(((methylsulfonyl)oxy)methyl)picolinates

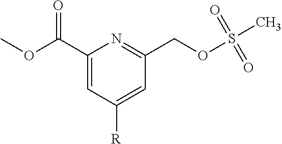

### R:

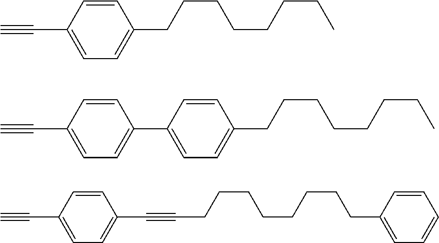

| | | | Empirical |
|---|---|---|---|
| | Structure | Nomenclature | formula |
| Example 9a | 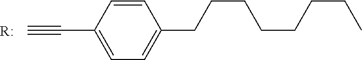 | methyl 6-(((methyl- sulfonyl)oxy)methyl)-4- ((4-octylphenyl) ethynyl)picolinate | C₂₅H₃₁NO₅S |
| Example 9b | 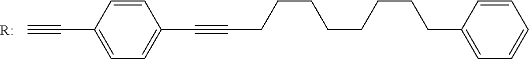 | methyl 6-(((methyl- sulfonyl)oxy)methyl)-4- ((4-(10-phenyldec-1-yn-1- yl)phenyl)ethynyl) | C₃₃H₃₅NO₅S |
| | | picolinate | |
| Example 9c | 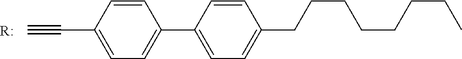 | methyl 6-(((methyl- sulfonyl)oxy)methyl)-4- ((4′-octyl-[1,1′-biphenyl]-4- yl)ethynyl)picolinate | C₃₁H₃₅NO₅S |

The intermediate obtained in example 5, 6 or 7 (1.64 mmol, 1 equiv.) is diluted in 18 ml of DCM (11 ml/mmol). The solution is conditioned under an inert atmosphere and is then cooled in a water/ice bath. The addition is carried out of triethylamine (3 equiv.) and of mesyl chloride (1.5 equiv.) dropwise. The reaction medium is stirred for 10 min and then 18 ml of saturated NaHCO₃ solution are added in order to bring the reaction to an end. The organic phase is recovered, dried over Na₂SO₄ and concentrated to dryness. A yellow solid is obtained which will be purified by flash chromatography on a silica cartridge with the heptane/AcOEt mixture.

The results are presented in the following table:

| | Example 9a | Example 9b | Example 9c |
|---|---|---|---|
| Molar mass | 457.59 | 557.71 | 533.68 |
| Yield | 97% | 97% | 63% |
| LCMS method | method 8 | method 4 | method 9 |
| RT | 8.1 min | 9.7 min | ND |
| m/z (ES+) | 458 | 558.31 | 534 |

a: NMR: ¹H NMR (60 MHZ, CDCl₃): δ 8.20 and 7.76 (m, 2H, pyridine), 7.48-7.24 (m, 9H, phenyl), 5.45 (s, 2H, CH₂OH), 4.04 (s, 3H, CH₃ ester), 3.19 (s, 3H, CH₃ mesyl), 2.75-2.36 (m, 4H, R—CH₂-phenyl and R—CH₂-alkyne), 1.40 (m, 12H, 6 CH₂ alkyl chain).

The following example 10 illustrates the alkylation reactions of the compound obtained in example 1c with the mesylated reactants described in example 9.

### EXAMPLE 10: Alkylation of the Intermediate Obtained in Example 1c (methyl 2-(3,6,9-triaza-1(2,6)-pyridinacyclodecaphan-3-yl)acetate)

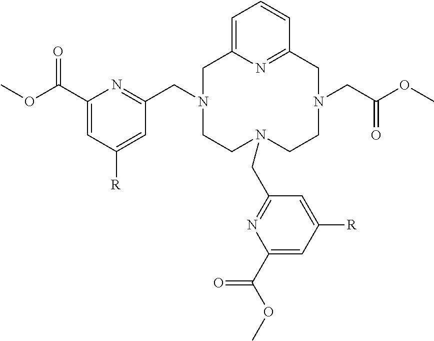

### R:

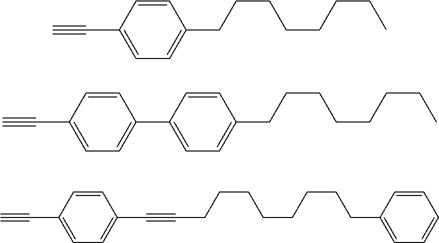

| | | | Empirical |
|---|---|---|---|
| | Structure | Name | formula |
| Example 10a | 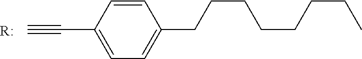 | dimethyl 6,6′-((9-(2- methoxy-2-oxoethyl)-3,6,9- triaza-1(2,6)-pyridina- cyclodecaphane-3,6- | C₆₂H₇₆N₆O₆ |
| | | diyl)bis(methylene))bis(4- | |
| | | ((4-octylphenyl)ethynyl) | |
| | | picolinate) | |
| Example 10b | 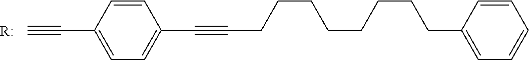 | dimethyl 6,6′-((9-(2- methoxy-2-oxoethyl)-3,6,9- triaza-1(2,6-pyridina- cyclodecaphane-3,6- | C₇₈H₈₄N₆O₆ |
| | | diyl)bis(methylene))bis(4- | |
| | | ((4-(10-phenyldec-1-yn-1- | |
| | | yl)phenyl)ethynyl)picolinate) | |
| Example 10c | 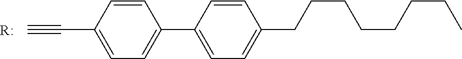 | dimethyl 6,6′-((9-(2- methoxy-2-oxoethyl)-3,6,9- triaza-1(2,6)-pyridina- cyclodecaphane-3,6- | C₇₄H₈₄N₆O₆ |
| | | diyl)bis(methylene))bis(4- | |
| | | ((4′-octyl-[1,1′-bipheny]-4- | |
| | | yl)ethynyl)picolinate) | |

The compound obtained in example 1c (methyl 2-(3,6,9-triaza-1(2,6)-pyridinacyclodecaphan-3-yl)acetate) (462 mg, 1.66 mmol, 1 equivalent) is diluted in 30 ml of anhydrous acetonitrile and then calcined calcium carbonate (2.5 equivalents) and the intermediate obtained in example 9 (9a or 9b or 9c) (2.1 equivalents) are added. The reaction medium is heated at 60° C. with stirring. The solution is subsequently cooled to ambient temperature and filtered. The salts are rinsed with acetonitrile and then the filtrate is concentrated to dryness. An orange oil is obtained which will be purified by flash chromatography with a silica cartridge with the DCM/MeOH mixture.

| | Example 10a | Example 10b | Example 10c |
|---|---|---|---|
| Mass | 1001.33 | 1201.57 | 1153.52 |
| Yield | 45% | 50% | 23% |
| TLC eluent | DCM/MeOH 8:2, | DCM/MeOH 9:1, | DCM/MeOH 8:2, |
| | Silica | Silica | Silica |
| | Rf = 0.78 | Rf = 0.38 | |
| LCMS method | Method 4 | Method 4 | Method 4 |
| RT | 16.1 min | 14.3 min | 14.4 min |
| m/z (ES+) | 1002 | 1202 | 1153.7 |

The following example 11 illustrates the production of the lipophilic ligands.

### EXAMPLE 11: Saponification of the Intermediates Obtained in Example 10, in Order to Obtain the Compounds of Formula (I)

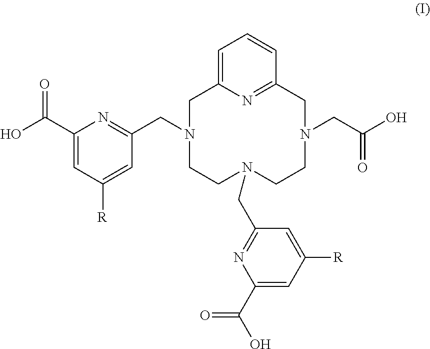

The groups R are chosen from:

### R:

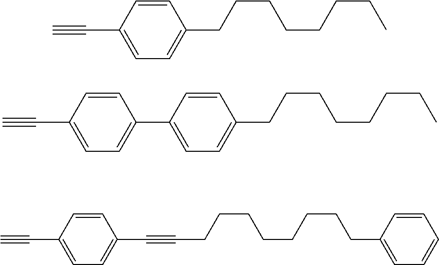

| Compounds | | | |
|---|---|---|---|
| of formula | | | |
| (I) | | | |
| according to | | | Empirical |
| the invention | Structure | Nomenclature | formula |
| Example 11a | 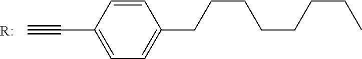 | 6,6′-((9-(carboxymethyl)- 3,6,9-triaza-1(2,6)-pyridina- cyclodecaphane-3,6- diyl)bis(methylene)) | C₅₉H₇₀N₆O₆ |
| | | bis(4-((4-octylphenyl) | |
| | | ethynyl)picolinic acid) | |
| Example 11b | 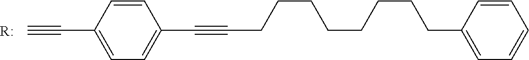 | 6,6′-((9-(carboxymethyl)- 3,6,9-triaza-1(2,6)-pyridina- cyclodecaphane- 3,6-diyl)bis(methylene)) | C₇₅H₇₈N₆O₆ |
| | | bis(4-((4-(10-phenyldec- | |
| | | 1-yl-1-yl)phenyl)ethynyl) | |
| | | picolinic acid) | |
| Example 11c | 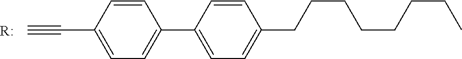 | 6,6′-((9-carboxymethyl)- 3,6,9-triaza-1(2,6)-pyridina- cyclodecaphane-3,6- diyl)bis(methylene))bis | C₇₁H₇₈N₆O₆ |
| | | (4-((4′-octyl-[1,1′-biphenyl]- | |
| | | 4-yl)ethynyl)picolinic acid) | |

The intermediate obtained in example 10 (10a or 10b or 10c) (1.00 mmol, 1 equivalent) is diluted in 10 ml of a 2M solution of potassium hydroxide in ethanol. The reaction medium is stirred at ambient temperature for 15 min. 15 ml of DCM are added and then the solution is cooled in a water/ice bath before adding metal-free 30% hydrochloric acid until a pH of 6-7 and a precipitate are obtained. The salts are filtered off and rinsed with DCM and then the filtrate is concentrated to dryness. A crude yellow solid is obtained which will be purified on a preparative column except for compound 11c, which will be left crude.

The fractions enriched in product of significance are combined, concentrated under vacuum and then lyophilized.

| | Example 11a | Example 11b | Example 11c |
|---|---|---|---|
| Molar mass | 959.25 | 1159.49 | 1111.44 |
| Yield | 41% (purified) | 26% (purified) | 42% (crude) |
| LCMS method | Method 4 | Method 4 | Method 9 |
| RT | 13.3 min | 13.93 min | 9.0 min |
| m/z (ES+) | 959.6 | 1159.7 | 1111.6 |

Examples 12, 13, 14 and 15 illustrate complexing reactions with different metals of significance.

### EXAMPLE 12: Synthesis of Complexes of Formula (III) According to the Invention

### Example 12a: Synthesis of the Metal (III) 6,6′-((9-(carboxylatomethyl)-3,6,9-triaza-1 (2,6)-pyridinacyclodecaphane-3,6-diyl)bis(methylene)bis(4-((4-(10-phenyldec-1-yn-1-yl)phenyl)ethynyl)picolinate) Complex

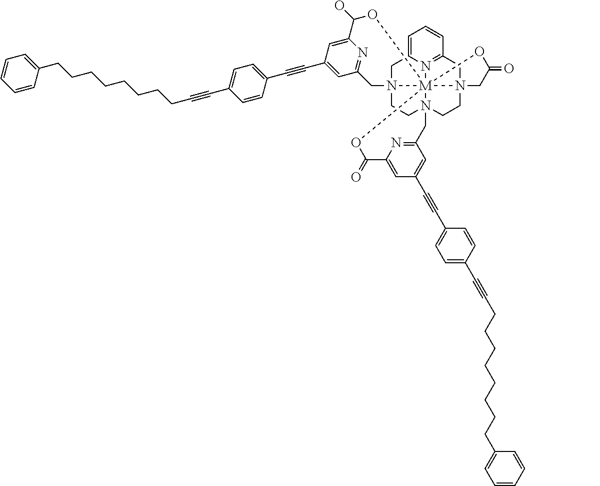

| Complexes of | | | | |
|---|---|---|---|---|
| formula (III) | | | | |
| according to | | | | |
| the invention | Reactant | M | Nomenclature | Empirical formula |
| Example 12a | YCl₃•6(H₂O) | ⁸⁹Y | Yttrium(III) 6,6′- | C₇₅H₇₅N₆O₆Y |
| | Strem Chemicals | | ((9-(carboxylatomethyl)- | |
| | Ref: 93-3903 | | 3,6,9-triaza- | |
| | | | 1(2,6)-pyridinacyclo- | |
| | | | decaphane-3,6- | |
| | | | diyl)bis(methylene))bis(4- | |
| | | | ((4-(10-phenyldec- | |
| | | | 1-yn-1- | |
| | | | yl)phenyl)ethynyl)picolinate) | |
| Example 12b | LuCl₃•6(H₂O) | Nat. Lu | Lutetium(III) | C₇₅H₇₅LuN₆O₆ |
| | Strem Chemicals | | 6,6′-((9-(carboxylatomethyl)- | |
| | Ref: 93-7111 | | 3,6,9- | |
| | | | triaza-1(2,6)- | |
| | | | pyridinacyclo- | |
| | | | decaphane-3,6- | |
| | | | diyl)bis(methylene))bis(4- | |
| | | | ((4-(10-phenyldec- | |
| | | | 1-yn-1- | |
| | | | yl)phenyl)ethynyl)picolinate) | |
| Example 12c | TbCl₃•6(H₂O) | ¹⁵⁹Tb | Terbium(III) | C₇₅H₇₅N₆O₆Tb |
| | Alfa Aesar | | 6,6′-((9-(carboxylatomethyl)- | |
| | Ref: 11210 | | 3,6,9- | |
| | | | triaza-1(2,6)- | |
| | | | pyridinacyclo- | |
| | | | decaphane-3,6- | |
| | | | diyl)bis(methylene))bis(4- | |
| | | | ((4-(10-phenyldec- | |
| | | | 1-yn-1-yl)phenyl)ethynyl)picolinate) | |

50 mg of ligand obtained in example 11b are dissolved in 13.3 ml of methanol and the metal hydrochloride hexahydrate (1.5 equiv.) is added to the medium. The pH of the solution is adjusted to 6 using a sodium methoxide solution (0.12M) and then the reaction medium is stirred at ambient temperature for 30 min. The solution is concentrated to dryness and then the crude solid is treated with 4 ml of dichloromethane. The solvent is then evaporated under reduced pressure in order to obtain a yellow solid.

| | Example 12a | Example 12b | Example 12c |
|---|---|---|---|
| Molar mass | 1245.37 | 1331.43 | 1315.39 |
| Yield | Quantitative | Quantitative | Quantitative |
| HPLC method | Method 7 | Infusion | Infusion |
| RT | 7.05 min | NA | NA |
| m/z (ES+) | 1246.6 | 1331.6 | 1315.5 |

### EXAMPLE 13: Synthesis of Radiolabeled Complexes

### Example 13a: Synthesis of the Yttrium-90 Complex of the Ligand Prepared in Stage 11b (6,6′-((9-(carboxymethyl)-3,6,9-triaza-1(2,6)-pyridinacyclodecaphane-3,6-diyl)bis(methylene))bis(4-((4-(10-phenyldec-1-yn-1-yl)phenyl)ethynyl)picolinic acid))

500 μl of solution of the compound obtained in example 11b (C=10⁻³ mol/l) are withdrawn and deposited in an SF8 glass vial, 500 μl of a buffered sodium acetate solution 3M pH 5.2 are added to a batch of approximately 629 MBq (17 mCi) of [⁹⁰Y] chloride ([⁹⁰Y] Cl) (PerkinElmer). The radioactive solution (˜500 μl) is then recovered and deposited in the vial containing the 500 μl of ligand solution. The mixture is stirred gently before being incubated at 80° C. for 20 min. At the end of the reaction, a sample is withdrawn using an insulin syringe and deposited on a thin layer chromatography plate (chromatography paper 1CHR, GE). The amount of radioactivity in the vial is measured using an ionization chamber (VDC-405 activimeter, model VIK202, used with the software v3.29; Comecer Netherlands, the Netherlands) precalibrated for the measurement of the radioactivity emitted by the disintegration of [⁹⁰Y] packaged in a glass vial and resuspended in a water/ethanol medium.

Extraction into Lipiodol® is subsequently carried out.

1 ml of physiological saline (Mini-Plasco NaCl 0.9%, BBraun), then 2 ml of Lipiodol® Ultra Fluid (Guerbet), are successively added to the reaction medium resulting from the radiolabeling contained in the vial and the latter is stirred vigorously manually using tongs in order to emulsify. The emulsion is subsequently broken by centrifugation (2600 g, 20 min) (Sigma 2-6, Fisher Bioblock Scientific), making it possible to obtain an oily lower phase, consisting of Lipiodol® (called “Lipiodolic phase”), containing the radiolabeled complex and an upper phase containing the water/ethanol mixture. The amount of radioactivity contained in the vial is measured using the same ionization chamber precalibrated for the measurement of [⁹⁰Y] in Lipiodol®. Most of the upper phase is sucked up with the syringe. The Lipiodolic phase is subsequently recovered using a 23G0, 60×25 mm needle (Sterican, BBraun) attached to a 1.0 ml syringe (1 ml Syringe Luer BD Plastipak, BD) in a preweighed SF8 glass vial.

The amount of radioactivity contained in the vials respectively containing the ethanol/ligand phase and the Lipiodolic phase is measured using the ionization chamber precalibrated for each of the measurement conditions. The vial containing the Lipiodolic phase is weighed using a precision balance (model TE64-0CE, Sartorius) making possible the calculation of the volume activity of the radiotracer.

### Calculations of the Synthesis Yields of the Radiotracer, of its Radiochemical Purity and of its Volume Activity:

### Synthesis Yield

The synthesis yield of the targeted radiotracer was established as having to be greater than or equal to 50%. It was calculated according to the following formula:

$$R = \frac{activity of the lipiodolic phase recovered}{engaged activity measured postincubation at 80 ° C .} \times 100 \%$$

### Radiochemical Purity (RCP) by Thin Layer Chromatography

The plates (chromatography paper 1CHR, GE) are eluted with a 0.1% by volume solution of triethylamine (Et₃N) in methanol (MeOH) until the migration front reaches the top of the sheet. The radioactivity present on the dried sheets is subsequently detected using an FLA-7000 phosphor imager operating with the acquisition software of the same name (version 1.1, FujiFilm). The RCP of the labeled radioligand was calculated using the Multi Gauge v3.1 program (FujiFilm), based on the quantification of the radioactivity detected on the sheet where the reaction medium migrated. In practice, this quantification is based on the creation of regions of significance plotted on each of the zones associated with the detected radioactivity. The RCP was calculated as follows:

$$RCP = \frac{radioactivity associated with the ligand ( PSL )}{\begin{matrix}radioactivity corresponding to the free \\ [ 90 Y ] present in the reaction medium ( PSL )\end{matrix}} \times 100 \%$$

### Calculation of the Volume Activity

The volume activity is determined by weighing the vial in which the Lipiodolic phase containing the radiotracer was recovered and by measuring the [⁹⁰Y] activity by means of an ionization chamber (the measured activity is corrected for the cosmic background noise and also for the physical decay of the [⁹⁰Y] (“background noise”). It will be calculated as follows, taking into account, as density of Lipiodol®, the value of 1.28 g/ml:

$$volume activity ( MBq / ml ) = specific activity ( MBq / g ) \times density ( Lipiodol ) ( g / ml )$$

with specific activity (MBq/g)=activity (lipiodolic phase) (MBq)/Lipiodol weight (g)
and Lipiodol® weight (g)=weight (full vial)−weight (empty vial, preweighed).

### Example 13b: Indium-111 Complex of the Ligand Obtained in Example 11b (6,6′-((9-(carboxymethyl)-3,6,9-triaza-1(2,6)-pyridinacyclodecaphane-3,6-diyl)bis(meth-ylene))bis(4-((4-(10-phenyldec-1-yn-1-yl)phenyl)ethynyl)picolinic acid))

The same protocol as that described in example 13a using a solution of indium-111 (Curium, 4.71 mCi, 174 MBq) results in the formation of the indium-111 complex.

### Example 13c: Lutetium-177 Complex of the Ligand Obtained in Example 11b (6,6′-(9-(carboxymethyl)-3,6,9-triaza-1(2,6)-pyridinacyclodecaphane-3,6-diyl)bis(meth-ylene))bis(4-((4-(10-phenyldec-1-yn-1-yl)phenyl)ethynyl)picolinic acid))

The same protocol as that described in example 13a using a solution of lutetium-177 (673 MBq) results in the formation of the lutetium-177 complex.

### Results of the Labeling of Lipiodol®

| | Example | Example | Example |
|---|---|---|---|
| | 13a | 13b | 13c |
| RadioChemical Purity (%) | 99.2 | 95.5 | 94.1 |
| Yield (%) | 99 | 94.5 | 87.4 |

### EXAMPLE 14: Yttrium-90 Complex of the Compound Obtained in Example 11c (6,6′-((9-(carboxymethyl)-3,6,9-triaza-1(2,6)-pyridinacyclodecaphane-3,6-diyl)bis(meth-ylene))bis(4-((4′-octyl-[^{1,1}′-biphenyl]-4-yl)ethynyl)picolinic acid))

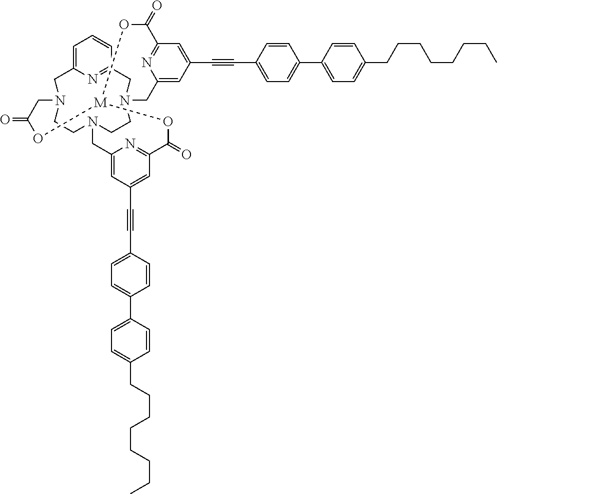

0.5 ml of yttrium-90 chloride in acetate buffer solution pH=5.2 is added to 0.5 ml of ligand obtained in example 11c dissolved in DMSO at a concentration of 10⁻³ mol/l. The solution is heated at 80° C. for 15 min. 1 ml of physiological saline+2 ml of Lipiodol® are added and the mixture is vigorously stirred. The phases are subsequently separated by centrifugation (4500 revolutions/min, 20 min) and the oily phase is collected in order to obtain the expected radiotracer.

| | |
|---|---|
| RCP (%) | 92.7 |
| Yield (%) | 89.5 |

### EXAMPLE 15: Yttrium-90 Complex of the Compound Obtained in Example 11a (6,6′-((9-(carboxymethyl)-3,6,9-triaza-1(2,6)-pyridinacyclodecaphane-3,6-diyl)bis(meth-ylene))bis(4-((4-octylphenyl)ethynyl)picolinic acid))

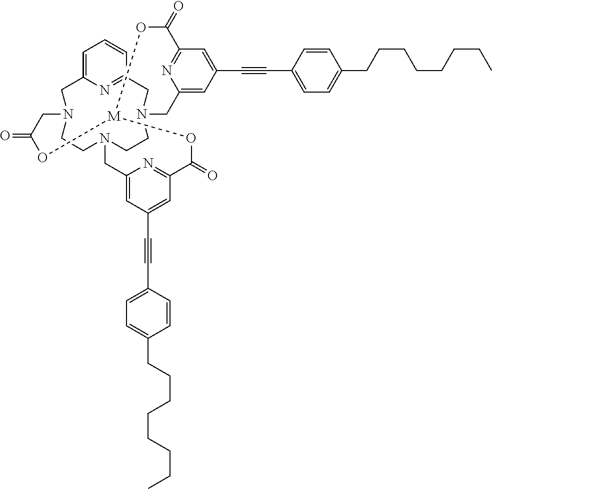

0.5 ml of yttrium-90 chloride in acetate buffer solution pH=5.2 is added to 0.5 ml of ligand obtained in example 11a dissolved in ethanol at a concentration of 10⁻³ mol/l. The solution is heated at 80° C. for 15 min. 1 ml of physiological saline+2 ml of Lipiodol® are added and the mixture is vigorously stirred. The phases are subsequently separated by centrifugation (3500 revolutions/min, 15 min) and the oily phase is collected in order to obtain the expected radiotracer.

| | |
|---|---|
| RCP (%) | 99.7 |
| Yield (%) | 92.9 |

### EXAMPLE 16: Stability Studies

1/with the Compounds According to the Invention:

The radiolabeled prototypes (branded Lipiodol®) are subsequently engaged in stability tests, consisting of an incubation period at 37° C. in the presence of physiological saline or human serum and kinetic monitoring of the passage of the radioactivity from the oily phase to the aqueous phase. These results are presented in the form of release curves indicating, on the axis of the abscissae, the incubation time (in hours) and, on the axis of the ordinates, the rate of release (% of the radioactivity transferred to the aqueous phase).

1 ml of freshly prepared radiotracer (examples 13a, 13b, 13c, 14, 15) is withdrawn and then deposited in a 12 ml flat-bottomed glass flask. The radioactivity is measured with an activimeter, and the time noted. 10 ml of physiological saline or human serum are added before stirring of the mixture. The flask is subsequently placed in the incubator at 37° C., with a stirrer set at 30 rpm.

Stirring is left for several days. The aqueous phase is withdrawn at different times in order to assay the yttrium-90 (radiolabeled complex of example 13a, example 14 and example 15), the indium-111 (radiolabeled complex of example 13b) or the lutetium-177 (radiolabeled complex of example 13c) released.

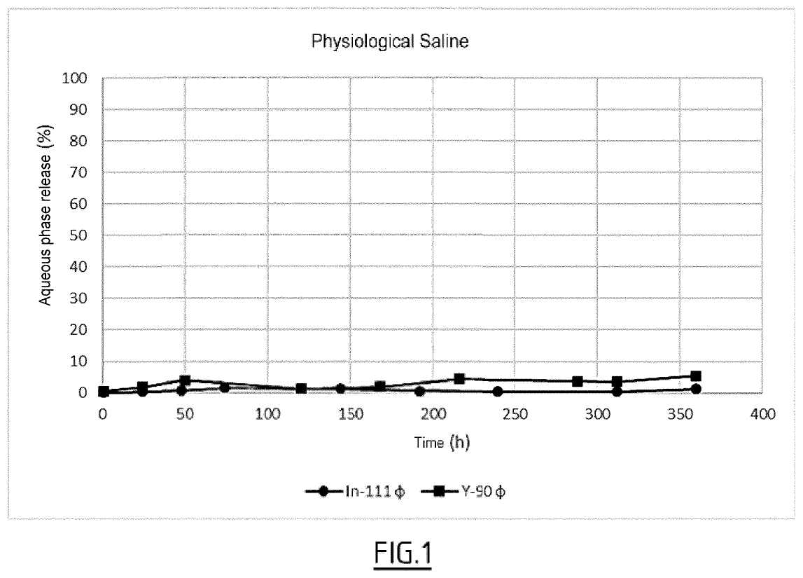

The results of the release of yttrium-90 or indium-111 for the compounds of examples 13a and 13b respectively into the aqueous phase (physiological saline) are presented in FIG. 1.

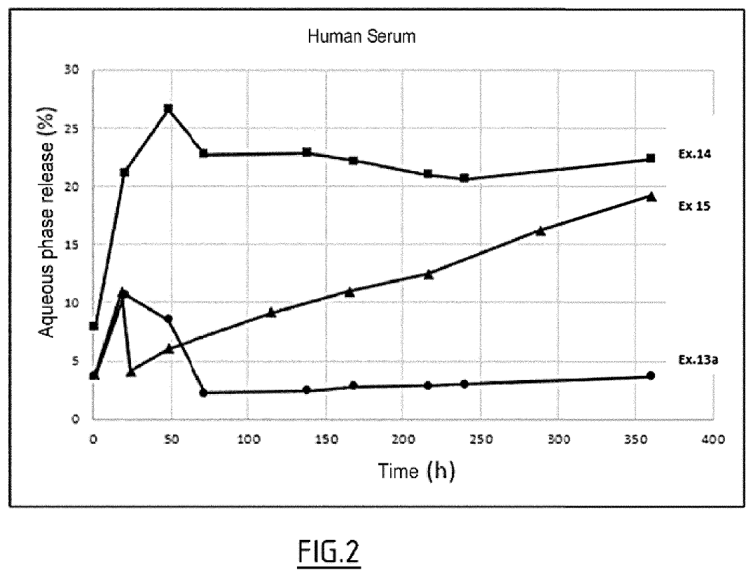

The results of the release of yttrium-90 into human serum (aqueous phase) for the compounds of examples 13a, 14 and 15 are presented in FIG. 2.

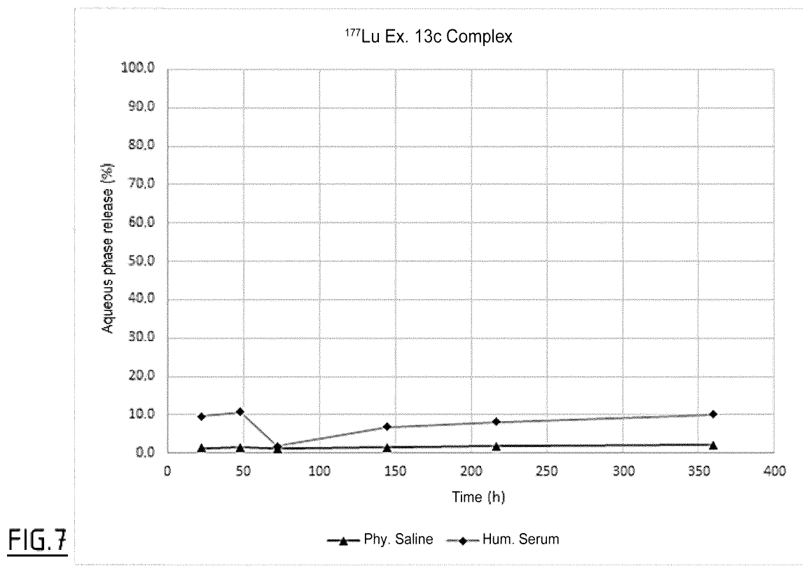

The results of the release of lutetium-177 into physiological saline and into human serum for the compound of example 13c are presented in FIG. 7. These results show that the lutetium-177 complex of example 13c exhibits a stability in salines/sera equivalent to that observed for the compounds of examples 13a and 13b (yttrium-90 complexes and indium-111 complex).

FIGS. 1, 2 and 7 show that the compounds which are subject matters of the invention are stable in physiological saline and human serum, that is to say that the radioactivity remains in the Lipiodolated phase and does not escape into the aqueous phase (less than 30% release for the compounds of examples 14 and 15 and less than approximately 10% release for the compounds of examples 13a, 13b and 13c), over a period of time of at least 350 h, which makes it possible to envisage their use in the radiotherapy of tumors of the liver.

2/with Comparative Compounds:

The following comparative compounds were studied under the same conditions in order to be able to compare their properties with those of the compounds of the invention.

### a) Structures of the Comparator Picolinates:

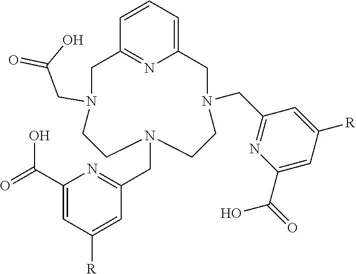

| | Comparator 1 | Comparator 2 |
|---|---|---|
| R | H | C≡CH—(CH₂)₉CH₃ |

| | Comparator 1 | Comparator 2 |
|---|---|---|
| Radiolabeling 1 | 98 | 98 |
| RCP (%) | | |
| Extraction yield 1 | 0 | 89.8 |
| (%) | | |
| Radiolabeling 2 | — | 98.9 |
| RCP (%) | | |
| Extraction yield 2 | — | 14.6 |
| (%) | | |
| Radiolabeling 3 | — | 69.7 |
| RCP (%) | | |
| Extraction yield 3 | — | 17 |
| (%) | | |

Comparator product 1, devoid of lipophilic residue on the picolinate units, is not extracted into Lipiodol®.

The results obtained with comparator product 2 indicate that the radiolabeling and the extraction into Lipiodol® are not reproducible for this substance.

These results contrast with those described in example 17 with radiolabelings which are perfectly reproducible for the compound described in example 11b.

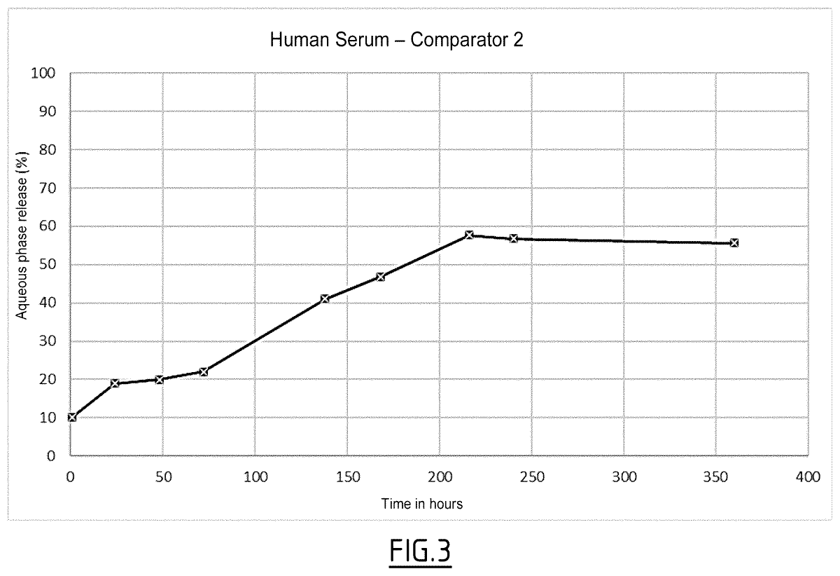

In addition, the stability of comparator 2 in human serum under the experimental conditions which are described above is shown in FIG. 3; it is significantly worse than that observed for the products according to the invention (FIG. 2).

This is because, in FIG. 3, it is found that more than 50% of the radioactivity has already escaped to the aqueous phase at T0+200 hours.

| | Compar- | Example | Example | Example |
|---|---|---|---|---|
| Compound | ator 2 | 13a | 14 | 15 |
| Escape of the | 58 | 3 | 21 | 12.5 |
| radioactivity into | | | | |
| the serum at 216 h (%) | | | | |

This indicates that the structure of the lipophilic residue R put onto the picolinate units is critical in obtaining equivalent properties (radiolabeling, reproducible and stability) to the compounds of the invention.

During the optimization of lipophilic residues, prototypes of related structure were prepared and tested. It is apparent that the structure of the group R had to be adjusted very finely in order to obtain compounds which are stable during the test carried out in physiological saline.

### b) Structures of Comparator Compounds 3, 4 and 5:

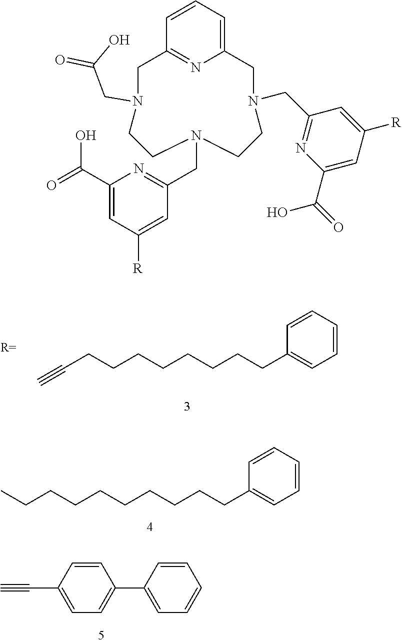

The data of extraction into Lipiodol® for the different compounds prepared are presented in the following table:

| | Comparator | Example | Comparator | Comparator | Example | Example |
|---|---|---|---|---|---|---|
| | 3 | 15 | 4 | 5 | 14 | 13a |
| Lipiodol ® | 51.0 | 92.9 | 93.4 | 84.5 | 89.5 | 90.0 |
| extraction (%) | | | | | | |

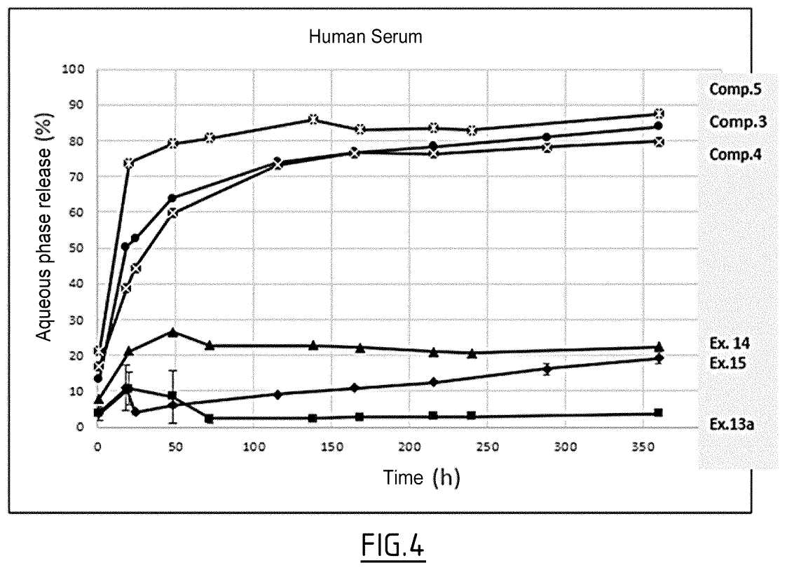

The stability data are presented in FIG. 4.

The stability curves of the compounds of the invention show a slower transfer of the radioactivity to the serum and a lower amplitude than the three comparator compounds. This is because none of these three comparator compounds is stable in the serum, that is to say that they exhibit rapid and significant escape of radioactivity toward the aqueous phase. This indicates that the structure of these compounds does not make it possible to obtain stable radiolabeled oily phases.

### EXAMPLE 17: Biodistribution Study on the Compound Obtained in Example 13a

The radiolabeling is carried out according to the procedure described in example 13a. Yttrium-90 (⁹⁰YCl in HCl) is supplied by PerkinElmer. The radiochemical purity is greater than 95%.

| | | | Radiochemical | | Specific | Volume |
|---|---|---|---|---|---|---|
| | | | yield | Activity | activity | activity |
| Time | | Batch | (%) | (MBq) | (MBq/g) | (MBq/ml) |
| 1 | h | [⁹⁰Y] T1 h | 96 | 226.6 | 157.7 | 201.9 |
| 24 | h | [⁹⁰Y] T3 h | 96 | 362.5 | 191.0 | 244.4 |
| D | 3 | [⁹⁰Y] T3 D | 96 | 350.6 | 159.5 | 204.2 |
| D | 6 | [⁹⁰Y] T6 D | 98 | 343.6 | 172.1 | 220.2 |

The quality of the radiolabeling obtained for each test indicates that the protocol is reproducible. The robustness of this radiolabeling has made it possible to produce radiopharmaceutical batches for in vivo studies.

56 female rats (Sprague-Dawley, aged from 8 to 10 weeks, weighing between 220 and 225 g, Janvier France) are acclimated for 5 days (23° C., humidity 22%) with free access to food and to drinking water.

Novikoff N1S1 cells (ATCC, UK) are used to induce hepatocellular carcinoma in the rats. Tumor induction consists in injecting the N1S1 cells into the rats according to the protocol described in the literature (Garin E., Denizot B., Roux J., et al., Description and technical pitfalls of a hepatoma model and of intra-arterial injection of radiolabeled Lipiodol in the rat, Lab Animals, 2005, 39, 314-320).

The radiolabeled complex of example 13a is injected via a cannula (26G) previously inserted into the hepatic artery of the rat. The injected dose is approximately 3.5 MBq.

The animals are sacrificed at 1 hour, 24 hours, 3 days and 6 days, and the blood and the various organs are removed and weighed after dissection.

The tubes containing the organs are counted using a gamma counter (PerkinElmer, USA) calibrated for yttrium-90.

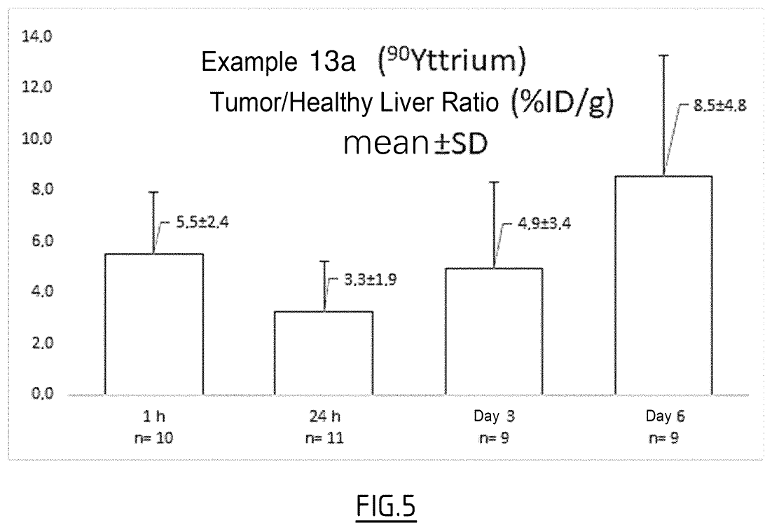

The results appear in the table below and in FIG. 5.

| | 1 h | 24 h | 3 D | 6 D |
|---|---|---|---|---|
| % Dose injected | 29 ± 10 | 17 ± 10 | 23 ± 14 | 35 ± 18 |
| into the tumor | | | | |
| Tumor/healthy | 5.4 ± 2.6 | 3.3 ± 1.9 | 4.9 ± 3.4 | 8.5 ± 4.8 |
| liver dose ratio | | | | |

### Distribution of the Radioactivity in the Liver

The study of the biodistribution shows that the product according to the invention is indeed captured by the tumor with a tumor/healthy liver ratio of at least 3 (see FIG. 5), and that the dose captured by the healthy liver is low because it is approximately 5% of the injected dose. This selectivity of the distribution for the tumor of the radioactive compound makes it possible to envisage its use in the radiotherapy of tumors of the liver.

The same experiments were carried out with the compound of example 13b (indium-111). The results show that a tumor/healthy liver injected dose ratio of greater than 5 is found at the time periods of 1 h and 6 days, which is consistent with the results observed for the compound of example 13a. This selectivity of the distribution for the tumor of the radioactive compound of example 13b makes it possible to envisage its use in the radiotherapy of tumors of the liver.

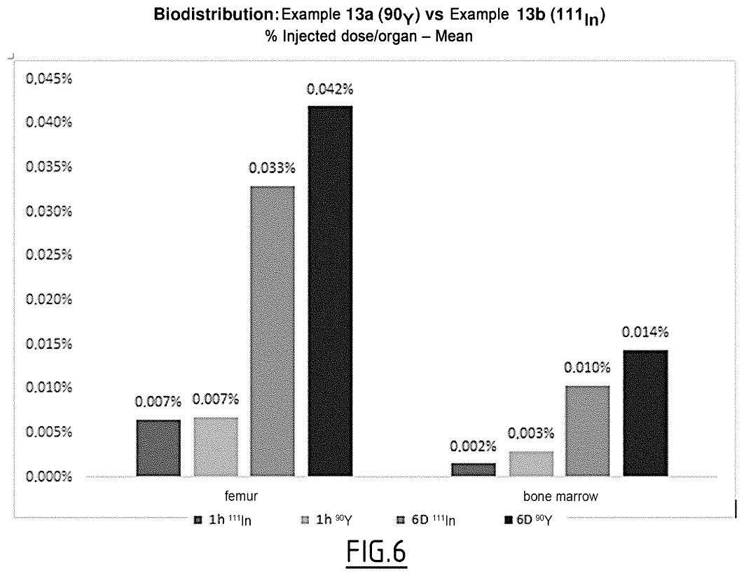

The percentages of dose injected into the femur and into the bone marrow for the compounds of example 13a and of example 13b, at 1 h and at 6 days after injection, are in FIG. 6. These results show that the residual dose of radioactivity in these organs is extremely low (less than 0.05%). These results of low doses captured by these sensitive organs make it possible to envisage the use of this compound in the radiotherapy of tumors of the liver.

1-14. (canceled)

15. A compound of the following general formula (I): 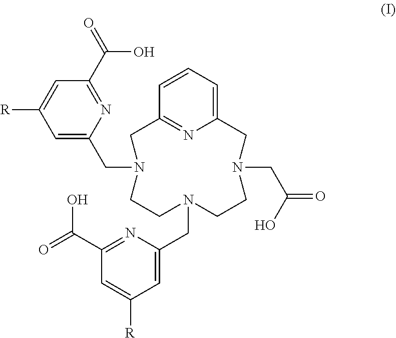
wherein R is a group of following formula (II):
—C≡C-Ph-L1-(CH₂)ₙ-L2 (II)
wherein:
L1 represents Ph, —C≡C—, or CH₂;
L2 represents H, Ph, or alkylphenyl; and
n is an integer in a range of 4 to 12;
or a pharmaceutically acceptable salt thereof.

16. The compound of claim 15 selected from the group consisting of the following: 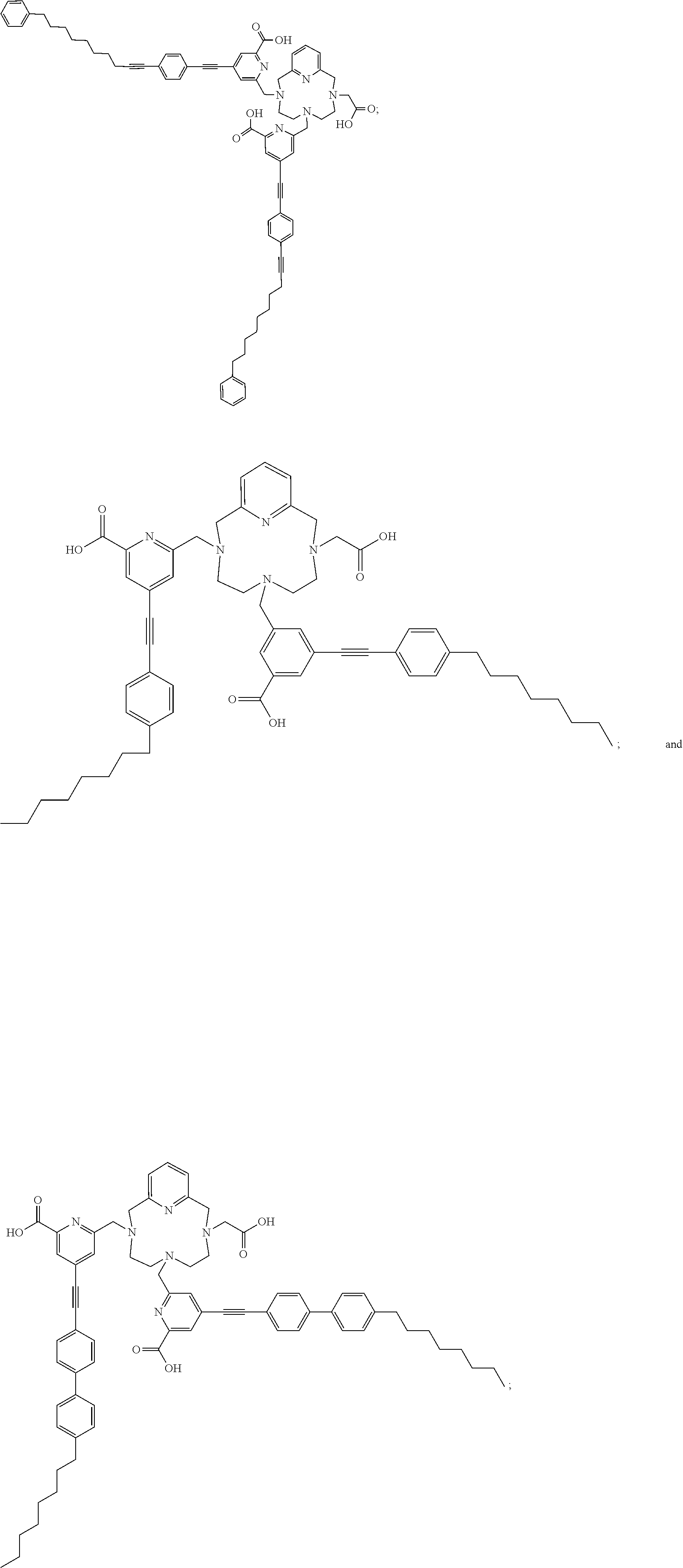
or a pharmaceutically acceptable salt thereof.

17. A complex comprising the compound of claim 15 and
a metallic element M.

18. The complex of claim 17, wherein M is a radioelement selected from the group consisting of ⁴⁴Sc(III), ⁴⁷Sc(III), ¹¹¹In(III), ¹⁵²Tb(III), ¹⁵⁵Tb(III), ¹⁴⁹Tb(III), ¹⁶¹Tb(III), ⁶⁴Cu(III), ⁶¹Cu(III), ⁶⁷Cu(II), ⁶⁸Ga(III), ⁹⁰Y(III), ¹⁵³Sm(III), ¹⁶⁶Ho(III), ¹⁷⁷Lu(III), ⁵²Mn, and ²²⁵Ac(III).

19. The complex of claim 17, wherein M is a radioelement selected from the group consisting of ⁹⁰Y(III), ¹⁷⁷Lu(III), ⁶⁷Cu(II), ²²⁵Ac(III), ¹¹¹In(III), ¹⁵²Tb(III), ¹⁵⁵Tb(III), ¹⁴⁹Tb(III), ¹⁶¹Tb(III) and ⁶⁸Ga(III).

20. The complex of claim 17 selected from the group consisting of the following: 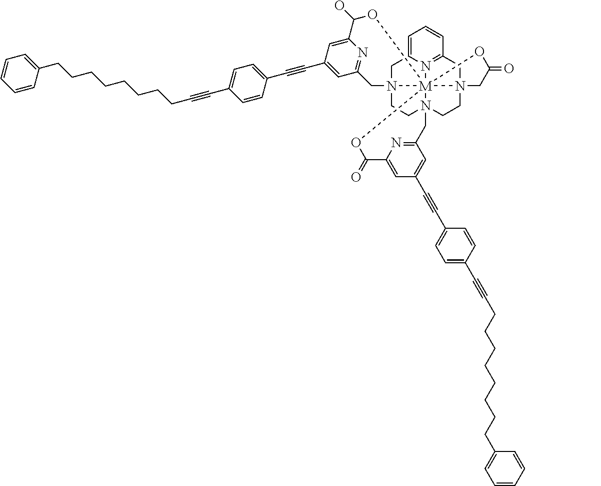
wherein M is ¹⁷⁷Lu(III), ⁹⁰Y(III) or ¹¹¹In(III); 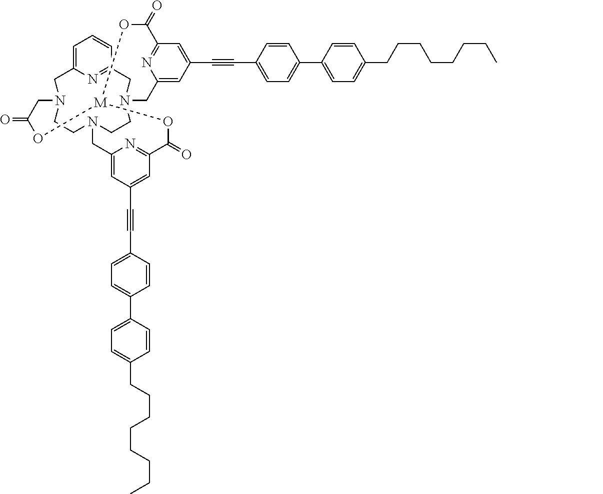
wherein M is ⁹⁰Y(III); and 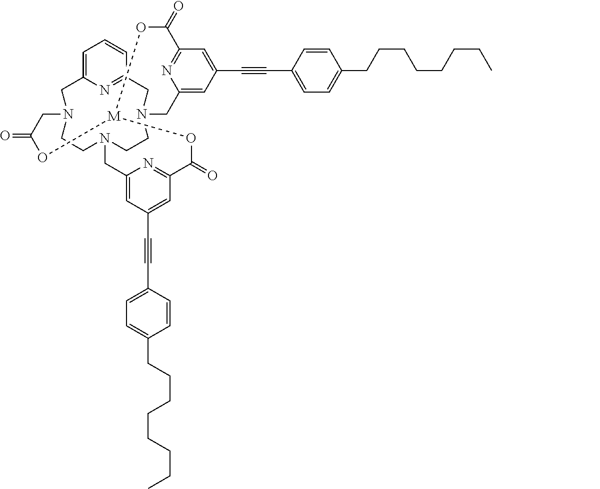
wherein M is ⁹⁰Y(III).

21. A pharmaceutical composition comprising the compound of claim 15 and one or more pharmaceutically acceptable excipient(s).

22. The pharmaceutical composition of claim 21, wherein at least one of the pharmaceutically acceptable excipient(s) is a radioprotector.

23. The pharmaceutical composition of claim 21 further comprising an iodinated oil.

24. The pharmaceutical composition of claim 23, wherein the iodinated oil comprises ethyl esters of iodinated fatty acids of poppy oil.

25. The pharmaceutical composition of claim 23, wherein the compound is the following:

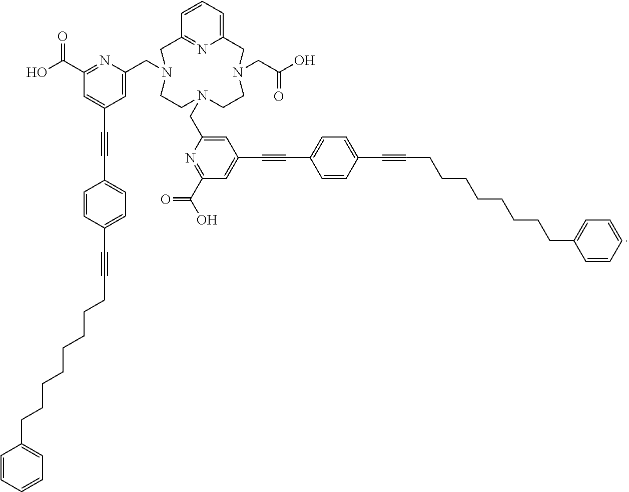 [SMILES: O=C(O)CN1CCN(Cc2cc(C#Cc3ccc(C#CCCCCCCCCc4ccccc4)cc3)cc(C(=O)O)n2)CCN(Cc2cc(C#Cc3ccc(C#CCCCCCCCCc4ccccc4)cc3)cc(C(=O)O)n2)Cc2cccc(n2)C1]

26. A pharmaceutical composition comprising the complex of claim 17 and one or more pharmaceutically acceptable excipient(s).

27. The pharmaceutical composition of claim 26, wherein at least one of the pharmaceutically acceptable excipient(s) is a radioprotector.

28. The pharmaceutical composition of claim 26 further comprising an iodinated oil.

29. The pharmaceutical composition of claim 28, wherein the iodinated oil comprises ethyl esters of iodinated fatty acids of poppy oil.

30. The pharmaceutical composition of claim 28, wherein the complex is the following:

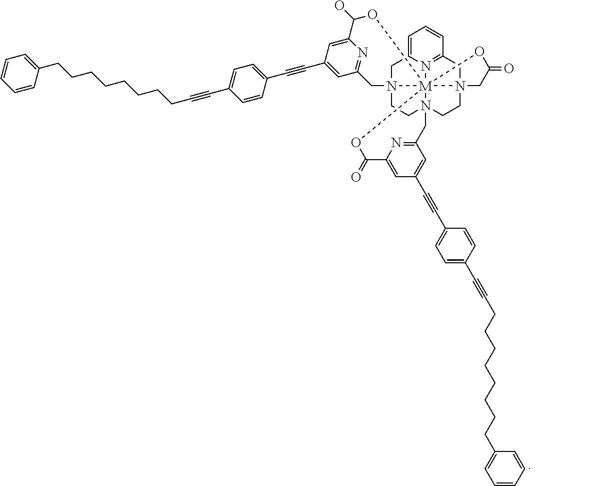

31. The pharmaceutical of claim 30, wherein M is ¹⁷⁷Lu(III) or ⁹⁰Y(III).

32. A method for treating a cancer in a mammal in need thereof, the method comprising administering a therapeutically effective amount of the complex of claim 17 to the mammal.

33. The method of claim 32, wherein the cancer is of the liver.

34. A method of medical imaging a patient, the method comprising administering an effective amount of the complex of claim 17 to the patient and conducting the medal imaging on the patient administered the complex.

32. A process for the preparation of a compound having a general formula (I): 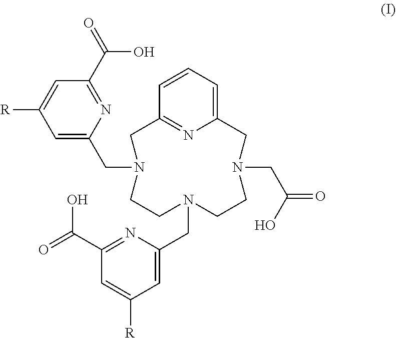
wherein R is a group of following formula (II):
—C≡C-Ph-L1-(CH₂)ₙ-L2 (II)
wherein:
L1 represents Ph, —C≡C—, or CH₂;
L2 represents H, Ph, or alkylphenyl; and
n is an integer in a range of 4 to 12;
the process comprising a stage of functionalization of a compound having a general formula (IX): 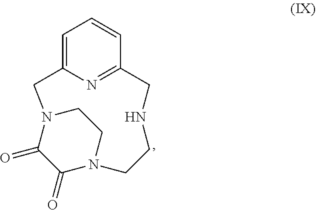
to form a compound of following general formula (X): 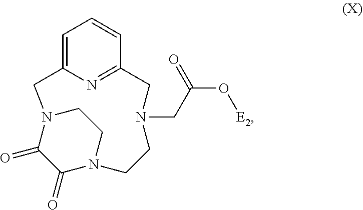
wherein E₂ is a C₁-C₄ alkyl protecting group selected from the group of methyl, ethyl, isopropyl, and tert-butyl.

33. The process of claim 32 further comprising:
a stage of deprotection of the compound of general formula (X) to obtain a compound having general formula (XI): 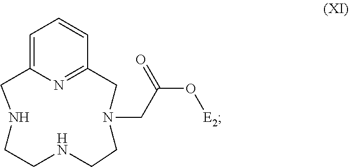
and
a stage of functionalization of the compound of general formula (XI) to obtain the compound of general formula (I) according to the following: 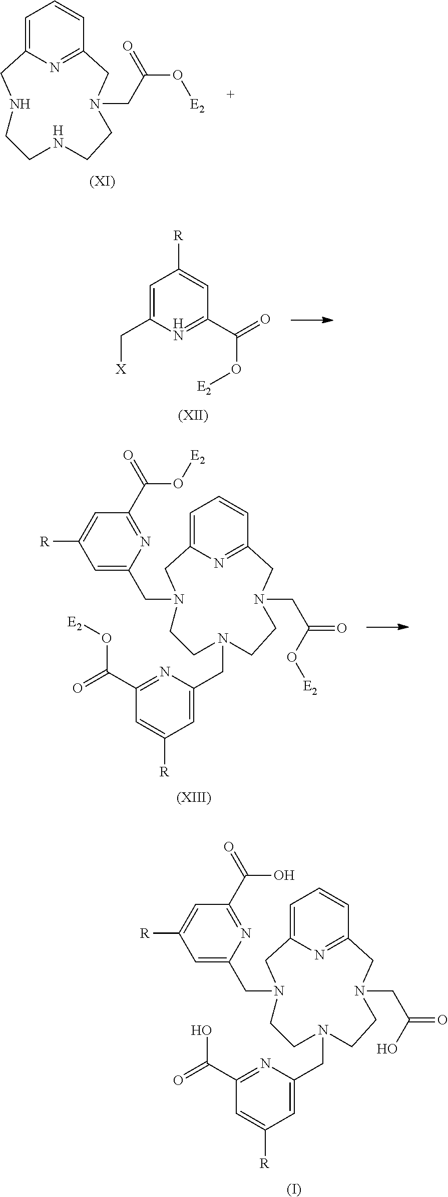
,
wherein E₁, E₂, and E₃ are each an independently selected C₁-C₄ alkyl protecting group selected from the group of methyl, ethyl, isopropyl, and tert-butyl.
